(19) United States
(12) Patent Application Publication
(10) Pub. No.: US 2019/0304595 A1
(43) Pub. Date: Oct. 3, 2019

(54) METHODS AND APPARATUS FOR HEALTHCARE TEAM PERFORMANCE OPTIMIZATION AND MANAGEMENT

(71) Applicant: General Electric Company, Schenectady, NY (US)

(72) Inventors: Ophira Bergman, San Diego, CA (US); Kirk Davis, San Diego, CA (US); Brian Shannon, San Diego, CA (US)

(21) Appl. No.: 15/943,124

(22) Filed: Apr. 2, 2018

(51) Int. Cl. G16H 40/20; G06Q 10/06; G06N 3/08; G06N 3/04

(52) U.S. Cl. CPC G16H 40/20; G06N 3/04; G06N 3/08; G06Q 10/06315

(57) ABSTRACT

Methods, apparatus, systems, and articles of manufacture are disclosed herein for healthcare processor optimization and management. An example apparatus includes a score prediction engine to determine a score for a previous resource composition, and predict, using machine learning techniques, resource composition scores for resource compositions different from the previous resource composition, and an output generator to generate results based on the resource composition scores, select a result from the results for interactive display, the selection based upon the resource composition scores, output the result for interaction via an interface, the interface receiving an input to accept the result/modify the result, and, in response to receiving the input, propagate the accepted result and/or the modified result to a server to, configure a first portion of the server with the result, reconfigure the first portion of the server with the result, and/or configure a second portion of the server with the result.

### FIELD OF THE DISCLOSURE

This disclosure relates generally to automated management systems and, more particularly, to methods and apparatus for healthcare team performance optimization and management.

### BACKGROUND

A variety of economic, technological, and administrative hurdles challenge healthcare facilities, such as hospitals, clinics, doctors' offices, etc., to provide quality care to patients. Economic drivers, less skilled staff, fewer staff, scheduling constraints, and complicated equipment create difficulties for effective management of resources (e.g., nurses, doctors, technicians, treatment rooms, equipment, etc.) to be deployed at healthcare facilities.

Further, healthcare provider consolidations create geographically distributed hospital networks in which higher level managers of healthcare providers may not work in the same building or even the same geographical region as the hospitals they are managing. At the same time, increasingly large amounts of data capable of assisting higher level managers in the day to day tasks of resource scheduling have become available. However, computer systems to aid in the analysis of these increasingly large amounts of data have yet to be developed.

### BRIEF DESCRIPTION OF THE DRAWINGS

FIG. 1A illustrates an example environment of use for an example resource director and inputs to the example resource director, by which the examples disclosed herein can be implemented.

FIG. 1B illustrates an example environment of use for the example resource director and outputs of the example resource director, by which the examples disclosed herein can be implemented.

FIG. 2A illustrates an example user interface for requesting a resource trade.

FIG. 2B illustrated an example user interface for accepting a resource trade.

FIG. 3 is a block diagram further detailing the example resource director of FIGS. 1A and 1B by which the examples disclosed herein can be implemented.

FIG. 4 is a block diagram further detailing an example score prediction engine of FIG. 3 by which the examples disclosed herein can be implemented.

FIG. 5 is an example data flow through an example neural network of FIG. 4.

FIG. 6 is a block diagram further detailing an example schedule optimizer of FIG. 3 by which the examples disclosed herein can be implemented.

FIG. 7 is a block diagram further detailing an example substitution recommender of FIG. 3 by which the examples disclosed herein can be implemented.

FIG. 8 is a block diagram further detailing an example resource manager of FIG. 3 by which the examples disclosed herein can be implemented.

FIGS. 9-11 illustrate example data flows and transformations of information flowing through the components of the example system of FIGS. 1A and 1B.

FIGS. 12-16 are flowcharts representative of machine readable instructions which can be executed to implement the resource director of FIG. 3.

FIG. 17 is a block diagram of an example processing platform structured to execute the instructions of FIGS. 12-16 to implement the resource director of FIG. 3.

The figures are not to scale. In general, the same reference numbers will be used throughout the drawing(s) and accompanying written description to refer to the same or like parts.

### BRIEF SUMMARY

Methods, apparatus, and articles of manufacture to facilitate improved resource scheduling and management based on patient outcome data and patient/employee satisfaction data using machine learning techniques are disclosed herein.

Certain examples provide an example apparatus to facilitate improved resource scheduling and management using machine learning techniques. The apparatus includes a memory to store instructions, and a processor to be particularly programmed using the instructions to implement at least a score prediction engine to process data from the memory to determine a score for a previous resource composition, and predict, using machine learning techniques, a plurality of resource composition scores, the plurality of resource compositions different from the previous resource composition, and an output generator to generate a plurality of results based on the plurality of resource composition scores, select a result from the plurality of results for interactive display, the selection based upon the plurality of resource composition scores, the result including at least one of a resource schedule, a resource substitution, or a resource ranking, output the result for interaction via an interface displayed with digital technology, the interface receiving an input to accept the result or modify the result, and in response to receiving the input via the interface, propagate at least one of the accepted result or the modified result to a scheduling server to, when the result includes the resource schedule, configure a first portion of the scheduling server with the result, when the result includes the resource substitution, reconfigure the first portion of the scheduling server with the result, and when the result includes the resource ranking, configure a second portion of the scheduling server with the result.

Certain examples provide an example computer readable medium to facilitate improved resource scheduling and management using machine learning techniques. The computer readable medium includes instructions that cause a machine to at least process data from a memory to determine a score for a previous resource composition, predict, using machine learning techniques, a plurality of resource composition scores, the plurality of resource compositions different from the previous resource composition, generate a plurality of results based on the plurality of resource composition scores, select a result from the plurality of results for interactive display, the selection based upon the plurality of resource composition scores, the result including at least one of a resource schedule, a resource substitution, or a resource ranking, output the result for interaction via an interface displayed with digital technology, the interface receiving an input to accept the result or modify the result, and propagate at least one of the accepted result or the modified result to a scheduling server to reconfigure one or more scheduling properties included with the server when the input is received.

Certain examples provide an example method to facilitate improved resource scheduling and management using machine learning techniques. The method includes processing data from a memory to determine a score for a previous resource composition, predicting, using machine learning techniques, a plurality of resource composition scores, the plurality of resource compositions different from the previous resource composition, generating a plurality of results based on the plurality of resource composition scores, selecting a result from the plurality of results for interactive display, the selection based upon the plurality of resource composition scores, the result including at least one of a resource schedule, a resource substitution, or a resource ranking, outputting the result for interaction via an interface displayed with digital technology, the interface receiving an input to accept the result or modify the result, and in response to receiving the input, propagating at least one of the accepted result or the modified result to a scheduling server to reconfigure one or more scheduling properties included with the server.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

While certain examples are described below in the context of medical or healthcare systems, other examples can be implemented outside the medical environment. For example, certain examples can be applied to non-medical resource management such as restaurant management, law firm management, sports management, etc.

In the field of hospital administration, a variety of economy, technological, and administrative hurdles challenge healthcare facilities, such as hospitals, clinics, doctors' offices, etc., to provide quality care to patients. Economic drivers, less skilled staff, fewer staff, scheduling constraints, and complicated equipment create difficulties for effective management of resources (e.g., nurses, doctors, technicians, treatment rooms, equipment, etc.) to be deployed at healthcare facilities.

Further, healthcare provider consolidations create geographically distributed hospital networks in which higher level managers of healthcare providers may not work in the same building or even the same geographical region as the hospitals they are managing. At the same time, increasingly large amounts of data capable of assisting higher level managers in the day to day tasks of resource scheduling have become available.

For example, more patient outcome data (e.g., patient never events (e.g., patient falls, death, wrong site surgery, bed sores, medication error, etc.), patient re-admits (e.g., number of re-admits, length of re-admits, frequency of re-admits, etc.), patient length of stay (e.g., number of total days, days in intensive care, number of therapy events during stay, etc.) is available to higher level managers than ever before. Additionally, digital satisfaction surveys have become more prevalently available in the hospital setting, providing higher level managers with yet another data stream to aid in efficiently and optimally managing hospital resources. However, determining correlations between these many data streams and the resources that are associated with the data streams can be an arduous if not impossible task for higher level managers.

A new solution, put forth by the methods, apparatus, and articles of manufacture disclosed herein, utilizes machine learning techniques to assist in hospital resource management.

Machine learning techniques, whether deep learning networks or other experiential/observational learning system, can be used to optimize results, locate an object in an image, understand speech and convert speech into text, and improve the relevance of search engine results, for example. While many machine learning systems are seeded with initial features and/or network weights to be modified through learning and updating of the machine learning network, a deep learning network trains itself to identify “good” features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

In the examples disclosed herein, the machine learning techniques are utilized to predict outcomes of various compositions of resources available to a hospital based upon previous outcomes of other compositions of resources available to the hospital. In some examples disclosed herein, the predicted outcomes can be utilized to generate a schedule for the resources available to the hospital (e.g., a schedule for nurses, a schedule for doctors, a schedule for equipment, a schedule for spaces, etc.). In other examples disclosed herein, the predicted outcomes can be utilized to determine allowable and/or optimal schedule substitutions in response to a scheduling event (e.g., a low census event, a high census event, a trade request, etc.). In yet other examples disclosed herein, the predicted outcomes can be utilized to determine a pool of the top resources available to the hospital (e.g., a high performing pool of the top nurses, a high performing pool of the top doctors, a high performing pool of the top pieces of equipment, etc.).

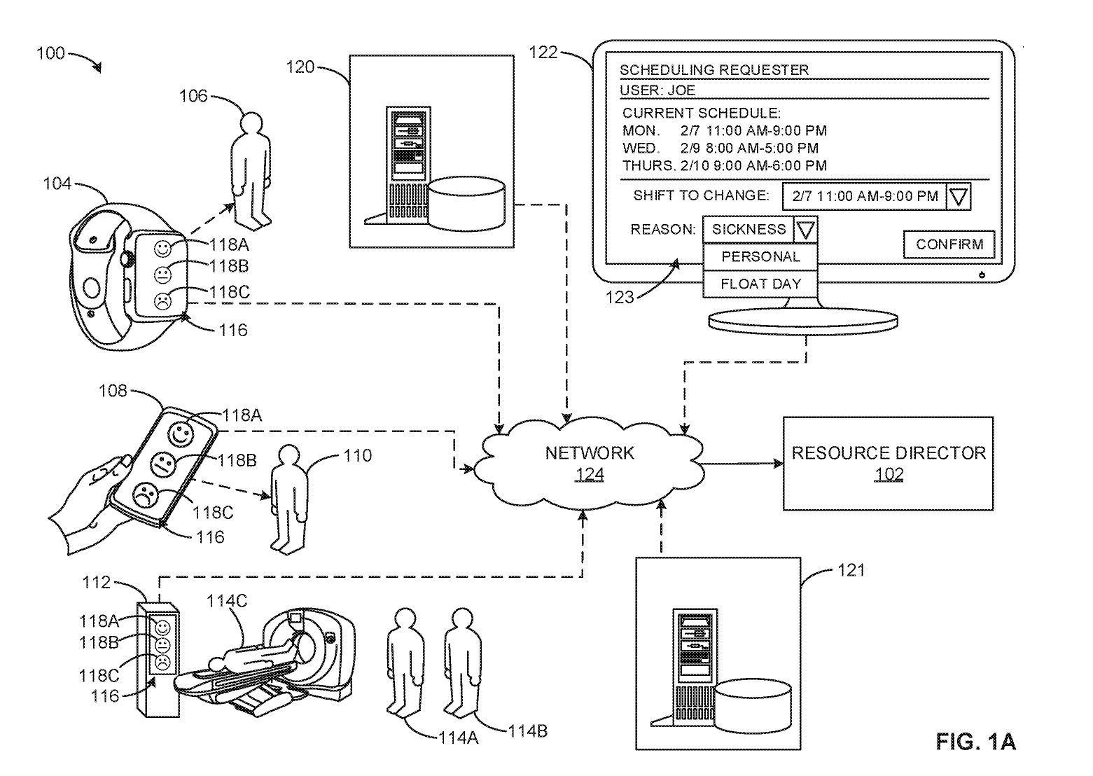

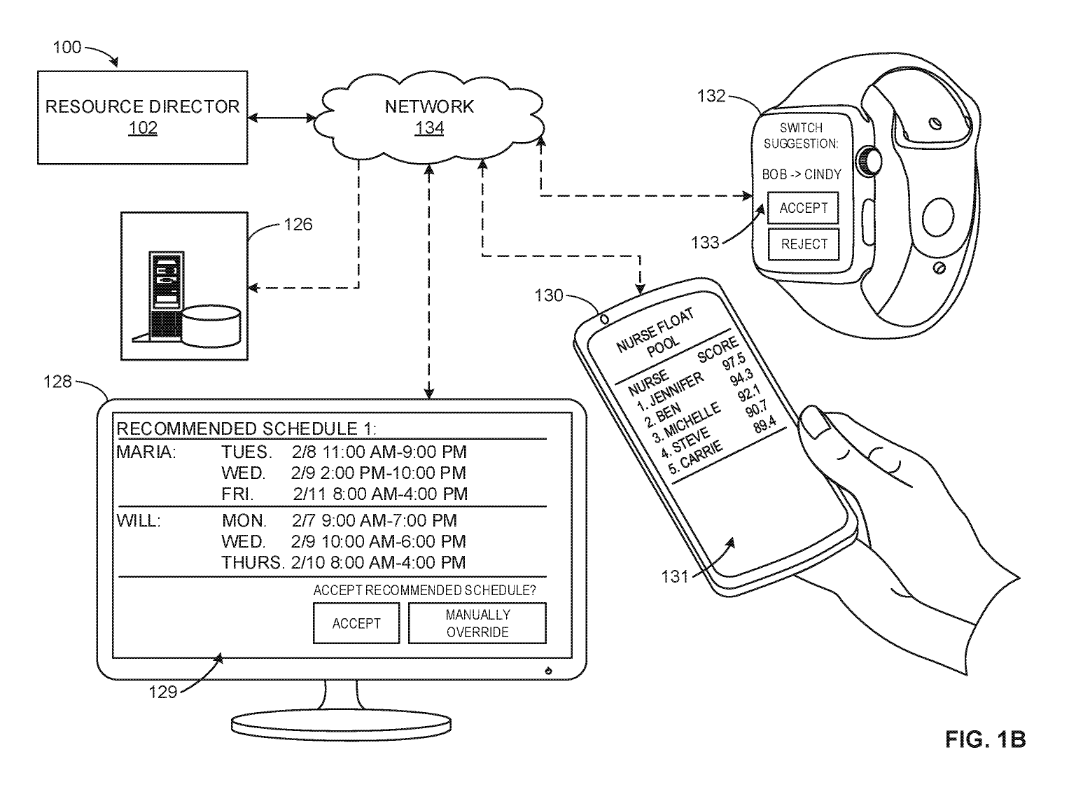

Turning now to the figures, FIG. 1A is a first view of an illustration of an example system 100 in which an example resource director 102 as disclosed herein can be implemented, the first view including one or more inputs to the example resource director 102. In the illustrated example of FIG. 1A, the inputs include a wearable 104 used by a user 106, a wireless device 108 used by a user 110, and a terminal 112 used by users 114A, 114B, and 114C. The users 106, 110, 114A, 114B, and 114C can be, but are not limited to, a patient, a family member or friend of the patient, a nurse, a doctor, hospital administrative staff, or any other individual in the physical proximity of a hospital utilizing the system 100. Additionally, while the terminal 112 is illustrated in a hospital room, the terminal 112 can be located at any position throughout the hospital (e.g., a hallway, a reception area, cafeteria, bathroom, etc.).

In some examples, each of the wearable 104, the wireless device 108, and the terminal 112 include, implement, or otherwise display a satisfaction survey 116. In the illustrated example of FIG. 1A, the satisfaction survey 116 includes three (3) tiers of satisfaction 118A-C: satisfied 118A, neutral 118B, and dissatisfied 118C. While in the illustrated example three (3) tiers of satisfaction 118A-C are shown, any number of tiers of satisfaction 118A-C greater than or equal to two (2) can be used.

In operation, any of the users 106, 110, 114A, 114B, and 114C can select one of the three (3) tiers of satisfaction 118A-C through the satisfaction survey 116. In response to a selection of one of the three (3) tiers of satisfaction 118A-C, the satisfaction survey is further to append a timestamp and a device identifier to the data point. Further, for the wearable 104 and the wireless device 108, an identity of a user (e.g., the user 106 for the wearable 104 and the user 110 for the wireless device 108, etc.) is known and/or can be determined (e.g., via facial recognition, fingerprint identification, retinal/iris scan, login, password, etc.) and can be appended to the data point.

Alternatively, for the terminal 112, any one of the users 114A, 114B, and 114C can complete the satisfaction survey 116 via the terminal 112. As such, the identity of the user for the terminal 112 is known such as based on a user identification number (ID), login and password/passcode, name entered at the terminal 112, etc. However, in some examples, based on a location of the terminal 112, the list of potential users may be limited. For example, if only user 114A had access to the room of the user 114C, the terminal 112 can determine the user can only be one of user 114A or 114C.

In some examples, the system 100 further includes a server 120 and a server 121. In the illustrated example of FIG. 1A, the server 120 hosts patient data and the server 121 hosts employee data. In some examples, the server 120 analyzes the patient data to identify, for example, patient never events (e.g., patient falls, death, wrong site surgery, bed sores, medication error, etc.), patient re-admits (e.g., number of re-admits, length of re-admits, frequency of re-admits, etc.), patient satisfaction, patient length of stay (e.g., number of total days, days in intensive care, number of therapy events during stay, etc.), and/or any other patient statistics or aggregate information that can be determined from the data. The patient data can also be correlated or processed with factors such as geodemographic data (e.g., a geographic home location of the patient, age of the patient, health history of the patient, etc.)

Looking to the server 121, employee data can be utilized to identify, for example, employee qualifications (e.g., employee certifications, employee qualification per unit, employee title/role, etc.), employee scheduling (e.g., an employee's normal hours, preferred shifts, scheduled vacation, etc.), employee reviews, and/or any other employee statistics or aggregate information that can be determined from the data. In some examples, the server 121 can store clock in and clock out times for employees which can, in some examples, include a flag denoting whether employees were on time for a shift. In such examples, the on time flag can further be processed by the server 121 to determine an on time percentage for each employee (e.g., the employee was more than 5 minutes late 10% of shifts, the employee has an on time rating of 95%, etc.). Further, the server 121 can store employee caseloads (e.g., one or more patients seen by the employee, determined from data stored in the server 120).

In some examples, the system 100 further includes a computing terminal 122. In the illustrated example of FIG. 1A, the computing terminal 122 interactively displays (e.g., on an interactive display) a scheduling request system 123 which can be used by employees of the hospital that implements the system 100. In the illustrated example of FIG. 1A, the scheduling request system 123 includes an input to select a shift to change, an input to select a reason for the change (e.g., sickness, personal day, float day, etc.), and a confirmation input. In the illustrated example of FIG. 1B, the computing terminal 122 displays the scheduling request system 123. The computing terminal 122 can additionally or alternatively display various other programs (e.g., resource management software, scheduling software, resource review software, etc.).

In the illustrated example of FIG. 1A, the resource director 102 connects to an example network 124. For example, the network 124 of the illustrated example of FIG. 1A is the Internet. However, the network 124 can be implemented using any suitable wired and/or wireless network(s) including, for example, one or more data buses, one or more Local Area Networks (LANs), one or more wireless LANs, one or more cellular networks, one or more private networks, one or more virtual private networks (VPNs), one or more public networks, etc. The network 124 enables the example resource director 102 to be in communication with at least one of the wearable 104, the wireless device 108, the terminal 112, the server 120, the server 121, and the computing terminal 122. As used herein, the phrase “in communication” including variances therefore, encompasses direct communication and/or indirect communication through one or more intermediary components and does not require direct physical (e.g., wired) communication and/or constant communication, but rather includes selective communication at periodic or aperiodic intervals as well as one-time events.

FIG. 1B is an illustration of a second view of the example system 100 in which an example resource director 102 as disclosed herein can be implemented, the second view including one or more outputs of the example resource director 102. In the illustrated example of FIG. 1B, the outputs include a server 126, a computing terminal 128, a wireless device 130, and a wearable 132.

The server 126, included in or otherwise implemented by the system 100, hosts resource scheduling data (e.g., the server 126 is a scheduling server, etc.) generated by the resource director 102. The scheduling data and scheduling properties hosted at the server 126 can include, but is not limited to, resource schedules, completed resource substitutions/switches, resource rankings, pools of high performing and low performing resources, and/or any other scheduling statistics or aggregate information. In some examples, the scheduling properties can be reconfigured (e.g., reconfigured scheduling properties) by an external input to the server 126. In some examples, the server 126 can further propagate the scheduling properties to one or more user devices.

The system 100 can further include the computing terminal 128. In the illustrated example of FIG. 1B, the computing terminal 128 displays a scheduling recommendation system 129 which can be used by employees (e.g., managers, etc.) of the hospital that implements the system 100. In the illustrated example of FIG. 1B, the scheduling recommendation system 129 includes an input to accept one of the schedules recommended by the resource director 102 in addition to an input to allow the manager to override (e.g., make a manual change, trigger an automated replacement, etc.) one of the schedules recommended by the resource director 102. In the illustrated example of FIG. 1B, the computing terminal 128 displays the scheduling recommendation system 129. The computing terminal 128 can additionally or alternatively display various other programs (e.g., resource management software, scheduling software, resource review software, etc.).

Additionally, the system 100 includes the wireless device 130. In the illustrated example of FIG. 1B, the wireless device 130 displays a resource pool 131 that can be used by employees (e.g., managers, etc.) of the hospital that implements the system 100. In the illustrated example of FIG. 1B, the resource pool 131 displays a pool of the top performing nurses, ranked by score. Additionally or alternatively, the resource pool 131 can display a pool of low-performing resources. Additionally or alternatively, the resource pool 131 can display a pool of any other hospital entity (e.g., doctors, support staff, equipment, etc.). In the illustrated example of FIG. 1B, the wireless device 130 displays the resource pool 131. The wireless device 130 can additionally or alternatively display various other programs (e.g., resource management software, scheduling software, resource review software, etc.).

The system 100 can further include the wearable 132. In the illustrated example of FIG. 1B, the wearable 132 displays a scheduling notifier 133 which can be used by employees (e.g., managers, etc.) of the hospital that implements the system 100. In the illustrated example of FIG. 1B, the scheduling notifier 133 displays a switch (e.g., a trade, a substitution, etc.) being proposed in addition to an input to accept the switch and an input to reject the switch. In some examples, the scheduling notifier 133 can further display a predicted impact on resource composition score as calculated by the resource director 102. In some examples, in response to receiving a reject input, the scheduling notifier 133 can further retrieve an additional suggestion from the resource director 102. In the illustrated example of FIG. 1B, the wearable 132 displays the scheduling notifier 133. The wearable 132 can additionally or alternatively display various other programs (e.g., resource management software, scheduling software, resource review software, etc.).

In the illustrated example of FIG. 1B, the resource director 102 connects to an example network 134. For example, the network 134 of the illustrated example of FIG. 1B is the Internet. However, the network 134 can be implemented using any suitable wired and/or wireless network(s) including, for example, one or more data buses, one or more LANs, one or more wireless LANs, one or more cellular networks, one or more private networks, one or more VPNs, one or more public networks, etc. The network 134 enables the example resource director 102 to be in communication with at least one of the server 126, the computing terminal 128, the wireless device 130, and the wearable 132. As used herein, the phrase “in communication” including variances therefore, encompasses direct communication and/or indirect communication through one or more intermediary components and does not require direct physical (e.g., wired) communication and/or constant communication, but rather includes selective communication at periodic or aperiodic intervals as well as one-time events.

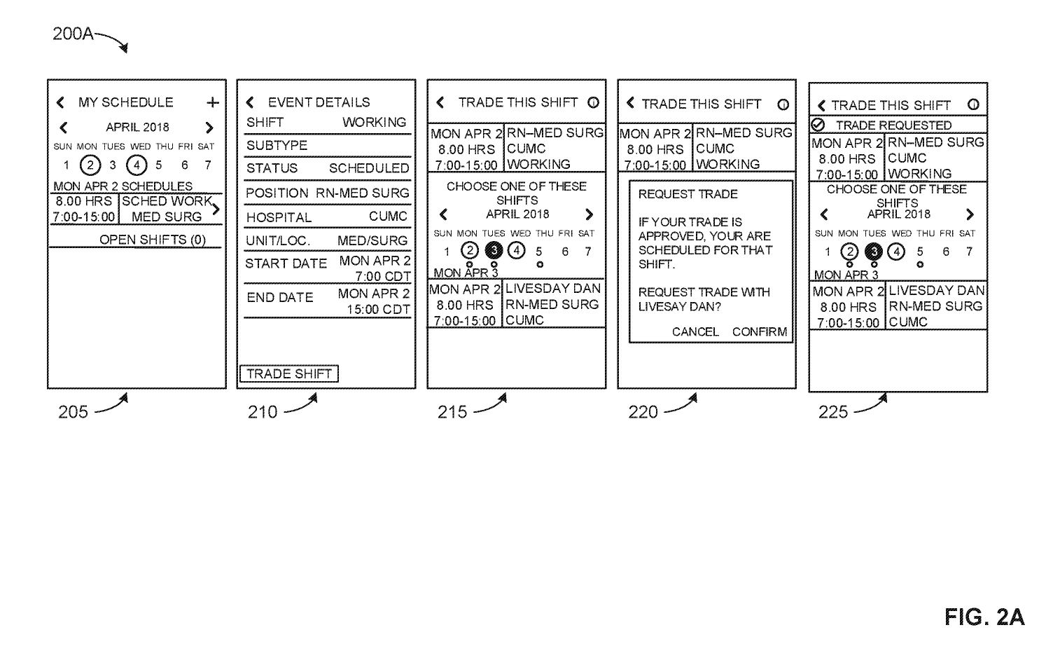

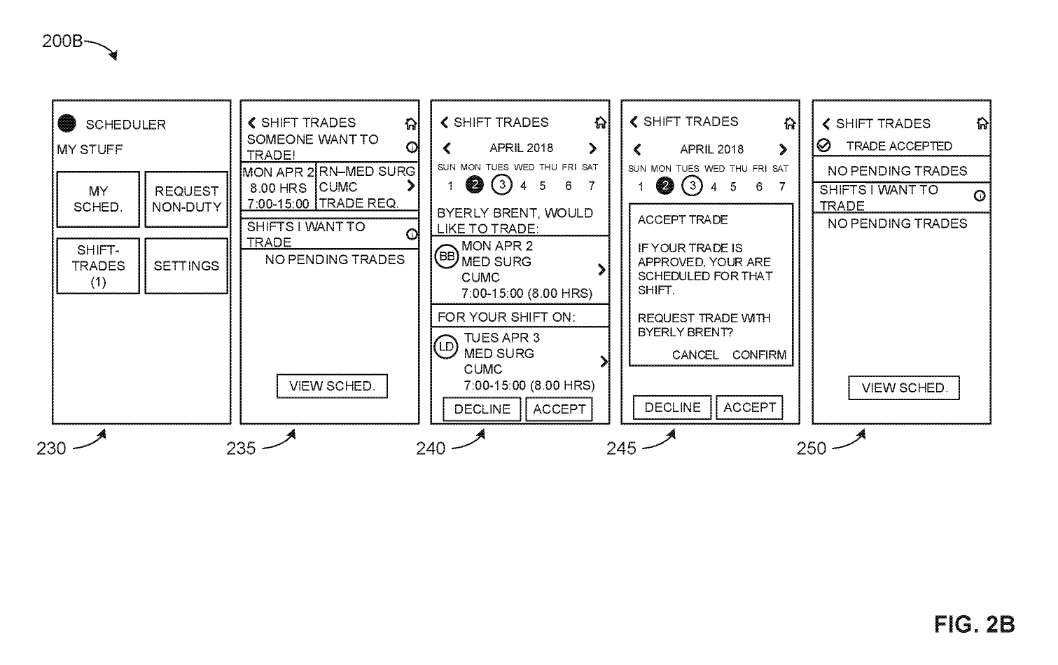

FIG. 2A is an illustrated example of a trade request interface 200a. The trade request interface, in the illustrated example of FIG. 2A, includes a first screen 205, a second screen 210, a third screen 215, a fourth screen 220, and a fifth screen 225.

The first screen 205 displays the current schedule of the user of the trade request interface 200a. For example, in the illustrated example of FIG. 2A, the user works an eight (8) hour shift from 7:00-15:00 on Monday, April 2^{nd}. Additional details of this shift are displayed on the second screen 210. For example, the illustrated shift is for an RN-Med Surg position at the CUMC hospital in the Med/Surg unit. Additionally, the second screen 210 includes an input to begin the trade request process.

In response to selecting the trade request input, the user is directed to the third screen 215 which, in the illustrated example of FIG. 2A, displays a list of possible shift trades. In some examples, using the methods disclosed herein and the resource director 102 of the illustrated example of FIG. 1A, the list of possible shift trades may only include those that impact a predicted score of the resource composition by a certain quantity. For example, the third screen 215 may only display those shift trades that would result in an increased predicted score of the resource composition. Additionally or alternatively, the third screen 215 may only display those shift trades that would result in a marginally decreased (e.g., decreased by 2%, 4%, etc.) predicted score of the resource composition.

In response to the user selecting one of the available shift trades, the user is directed to the fourth screen 220 and, in response to confirming the trade request, the user is further be directed to the fifth screen 225.

FIG. 2B is an illustrated example of a trade acceptance interface 200b. The trade acceptance interface 200b, in the illustrated example of FIG. 2B, includes a sixth screen 230, a seventh screen 235, an eighth screen 240, a ninth screen 245, and a tenth screen 250.

The sixth screen 230, displayed to the user upon start-up of the trade acceptance interface 200b, displays a menu including a tab for viewing received shift trade requests. In the illustrated example of FIG. 2B, one shift trade request has been received.

Upon selecting the received shift trade requests tab, the seventh screen 235 is displayed to the user. The seventh screen 235 displays a list of received shift trade requests in addition to a list of distributed shift trade requests. In the illustrated example of FIG. 2B, the user has received one (1) shift trade request and has not requested any shift trades.

In response to selecting the received shift trade request, the user is directed to the eighth screen 240 which displays details of the shift trade request. In the illustrated example of FIG. 2B, the shift trade request includes trading the user's shift on Tuesday Apr. 3, 2018 for another individual's shift on Monday Apr. 2, 2018. The eighth screen 240 further includes inputs to at least one of accept or decline the trade request.

In response to the user selecting the accept input, the ninth screen 245 is displayed to the user. The ninth screen 245 allows the user to confirm the trade acceptance and, in response to the confirmation, the tenth screen 250 is displayed to user. The tenth screen 250, in the illustrated example, is the same portion of the interface as the seventh screen 235. However, as the user previously accepted the one outstanding trade request shown on the seventh screen 235 at the eighth screen 240 and the ninth screen 245, the tenth screen 250 does not show any shift trade requests.

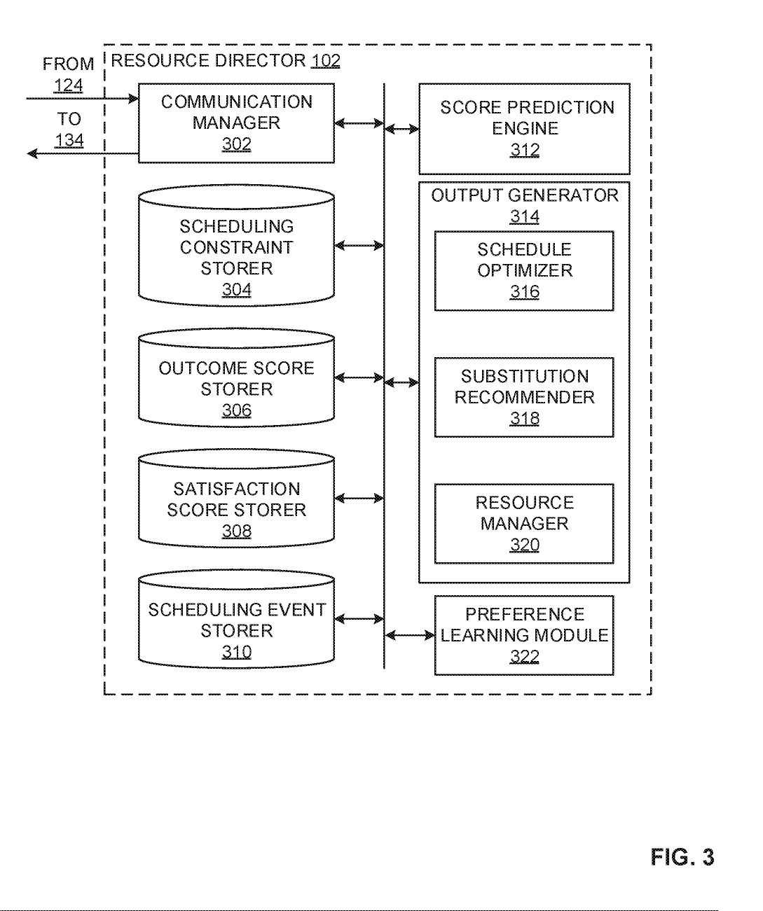

FIG. 3 is a block diagram of an example implementation of the example resource director 102 of FIGS. 1A and 1B. The resource director 102, in some examples, can include an example communication manager 302, an example scheduling constraint storer 304, an example outcome score storer 306, an example satisfaction score storer 308, an example scheduling event storer 310, an example score prediction engine 312, an example output generator 314 which can, in some examples, include an example schedule optimizer 316, an example substitution recommender 318, and an example resource manager 320, and an example preference learning module 322.

The communication manager 302, included in or otherwise implemented by the resource director 102, can at least one of transfer data to and receive data from at least one of the wearable 104, the wireless device 108, the terminal 112, the server 120, the server 121, the computing terminal 122, the server 126, the computing terminal 128, the wireless device 130, and the wearable 132 via at least one of the network 124 and the network 134.

In some such examples, the communication manager 302 can be implemented by any type of interface standards, such as an Ethernet interface, a universal serial bus (USB), and/or a PCI express interface. Further, the interface standard of the communication manager 302 is to at least one of match the interface of the network 124 and the network 134 or be converted to match the interface of the network 124 and the network 134.

The scheduling constraint storer 304, included in or otherwise implemented by the resource director 102, can store scheduling constraints as retrieved from the server 121. In some examples, the scheduling constraints stored by the scheduling constraint storer 304 can include, but are not limited to, hard constraints such as safe scheduling constraints including consecutive hour limits, maximum hour limits per seven day period, and maximum number of consecutive shifts, overtime rules including hours per day and hours per week, union rules including rotation of staff, hour limits, mandatory staff rotation, etc., shift overlap conflicts, indicated availability including FMLA leave, scheduled PTO, scheduled training, and/or call ins for sickness, and employee status (e.g., per diem, full time, part time, etc.). Additionally, the scheduling constraint storer 304 can include soft constraints such as scheduling preferences.

The outcome score storer 306, included in or otherwise implemented by the resource director 102, can store patient data received from the server 120. For example, the outcome score storer 306 can store patient never events (e.g., patient falls, death, wrong site surgery, bed sores, medication error, etc.), patient re-admits (e.g., number of re-admits, length of re-admits, frequency of re-admits, etc.), patient length of stay (e.g., number of total days, days in intensive care, number of therapy events during stay, etc.), and/or any other patient statistics or aggregate information that can be determined from the data. Further, based on data retrieved from the server 121, the outcome score storer 306 can store identities of employees (e.g., nurses, doctors, technicians, etc.) associated with (e.g., had an interaction with) the patient.

The satisfaction score storer 308, included in or otherwise implemented by the resource director 102, can store satisfaction data received from the satisfaction survey 116 that is included in, implemented by, and/or otherwise displayed on at least one of the wearable 104, the wireless device 108, and/or the terminal 112 via the network 124, as illustrated in FIG. 1A. In some examples, the satisfaction data can be stored in conjunction with a timestamp associated with completion of the satisfaction survey 116 and an identifier of the device (e.g., the wearable 104, the wireless device 108, the terminal 112, etc.) on which the satisfaction survey 116 was completed. In some examples, if the satisfaction survey 116 was completed on the wearable 104 or the wireless device 108, an identify of the user (e.g., the user 106 and the user 110) who completed the satisfaction survey 116 can be stored in conjunction with the satisfaction data.

In some examples, based on patient data retrieved from the server 120 and caseload data retrieved from the server 121, other individuals associated with the user who completed the satisfaction survey 116 (e.g., a patient's doctor, a patient's nurse, a patient's family member, a doctor's assistant, a nurse's hospital tech, etc.) can be stored in conjunction with the satisfaction data.

The scheduling event storer 310, included in or otherwise implemented by the resource director 102, can store scheduling requests received from the computational terminal 122, via the network 124. For example, as illustrated in FIG. 1A., the scheduling event storer 310 can store a notification that Joe is requesting off of his 2/7 shift, lasting from 11:00 AM until 9:00 PM for sickness. Additionally or alternatively, the scheduling event storer 310 can store census data (e.g., the current patient population and/or the expected future patient population of the hospital) as retrieved from the server 120 via the network 124 as illustrated in FIG. 1A. Additionally or alternatively, any data that would require and/or recommend a modification to the resource schedule can be stored in the scheduling event storer 310.

At least one of the scheduling constraint storer 304, the outcome score storer 306, the satisfaction score storer 308, and the scheduling event storer 310 can be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). At least one of the scheduling constraint storer 304, the outcome score storer 306, the satisfaction score storer 308, and the scheduling event storer 310 can additionally or alternatively be implemented by one or more double data rate (DDR) memories such as DDR, DDR2, DDR3, mobile DDR (mDDR), etc. At least one of the scheduling constraint storer 304, the outcome score storer 306, the satisfaction score storer 308, and the scheduling event storer 310 can additionally or alternatively be implemented by one or more mass storage devices such as hard disk drive(s), compact disk drive(s), digital versatile disk drive(s), etc. While in the illustrated example each of the scheduling constraint storer 304, the outcome score storer 306, the satisfaction score storer 308, and the scheduling event storer 310 are illustrated as a single database, any of one the scheduling constraint storer 304, the outcome score storer 306, the satisfaction score storer 308, and/or the scheduling event storer 310 can be implemented by an number and/or type(s) of databases. Further, the data stored in the scheduling constraint storer 304, the outcome score storer 306, the satisfaction score storer 308, and the scheduling event storer 310 can be in any format such as binary data, comma delimited data, tab delimited data, structured query language (SQL) structures, etc.

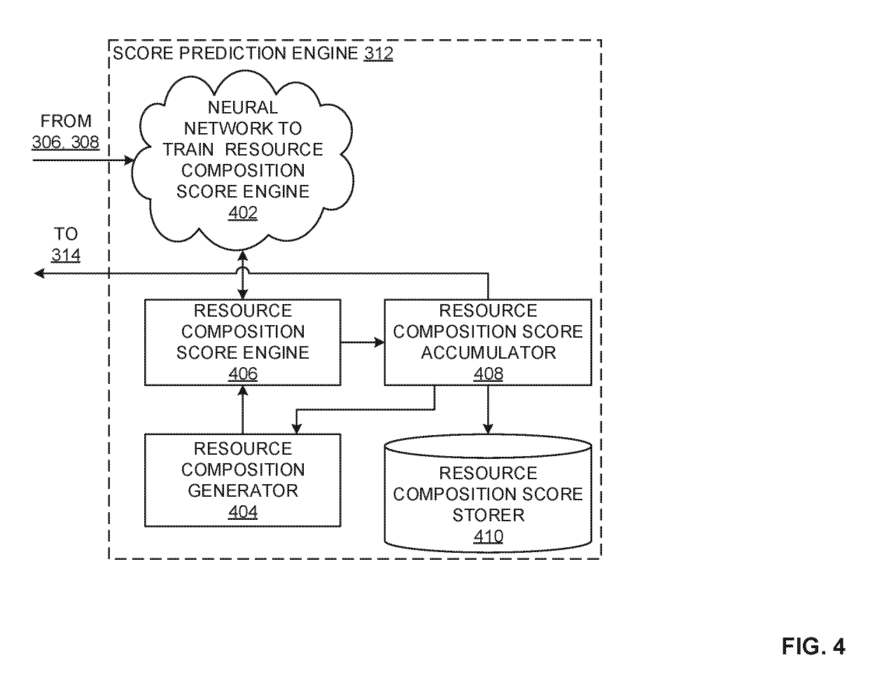

The score prediction engine 312, included in or otherwise implemented by the resource director 102, detailed further in conjunction with FIG. 4, can utilize a set of previously recorded data for one or more combinations of resources to determine a predicted outcome for one or more other combinations of resources. In some examples, an output of the score prediction engine 312 is further to be distributed to the example output generator 314.

In some examples, the output generator 314, included or otherwise implemented by the resource director 102, generates an output based on data received from the score prediction engine 312. In general, the output generator 314 receives a plurality of scheduling options from the score prediction engine 312 and determines a portion of the plurality of scheduling options to present to a user, the portion selected based on one or more selected criteria. For example, the output generator 314 can receive 10,000 possible schedules from the score prediction engine 312 and select the 3 highest performing schedules to present to the user.

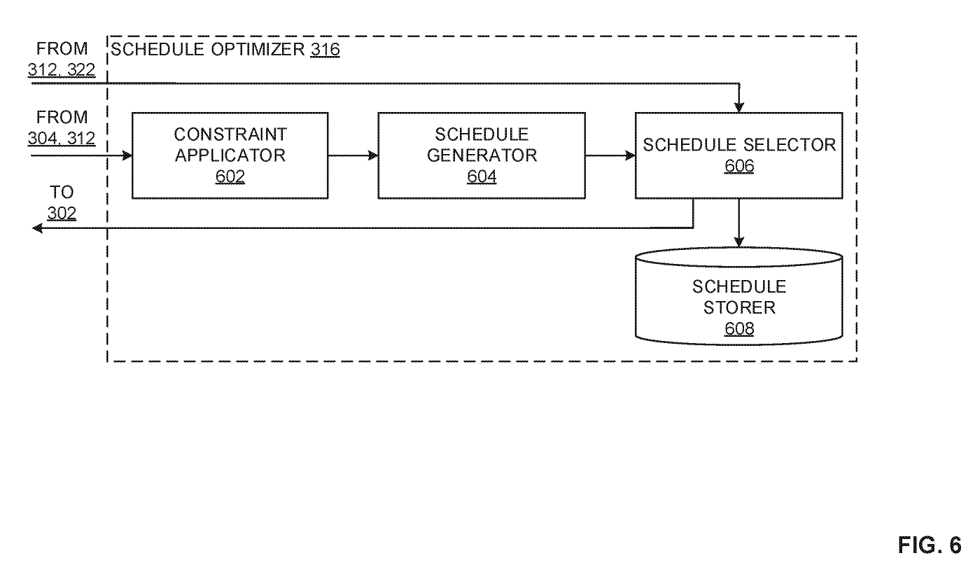

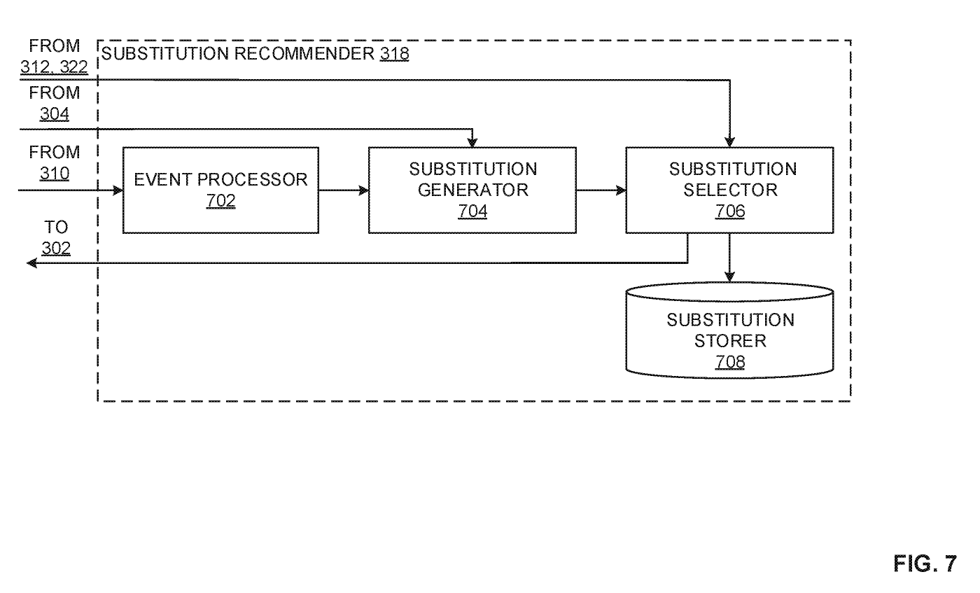

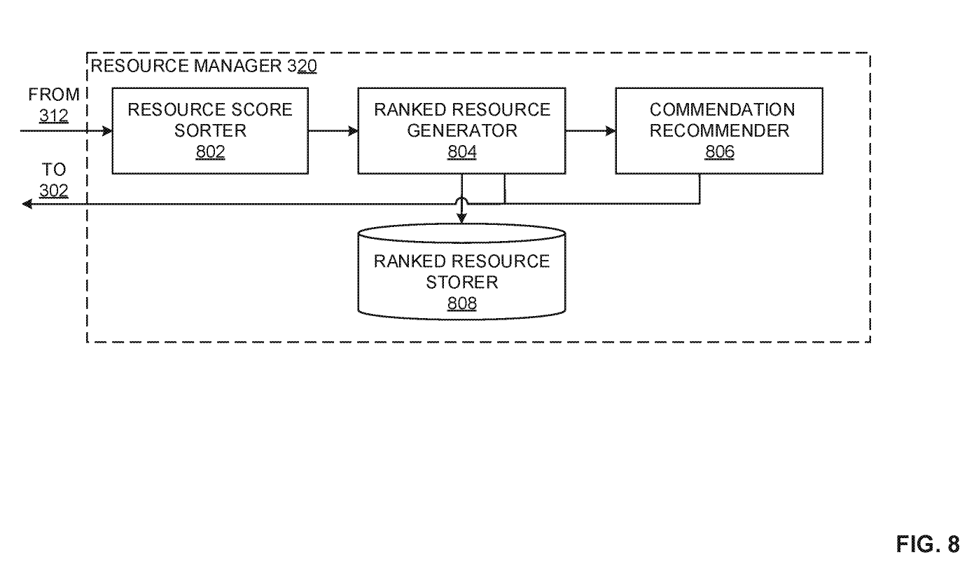

In some examples, the output generator 314 includes the example schedule optimizer 316, which can generate a resource schedule and is further detailed in conjunction with FIG. 6, the example substitution recommender 318, which can determine one or more recommended resource substitutions based on a substitution request and is further detailed in conjunction with FIG. 7, and the example resource manager 320, which can determine a pool (e.g., a group) of the highest rated resources and is further detailed in conjunction with FIG. 8.

In some examples, in response to at least one of the schedule optimizer 316, the substitution recommender 318, and the resource manger 320 completing generation of an output (e.g., a resource schedule, a resource substitution recommendation, a top resource pool, etc.), the output generator 314 is further to distribute the output along with a destination device tag for the output to the communication manager 302. Utilizing the destination device tag, the communication manager 302 is further to distribute the output to at least one of the server 126, the computer terminal 128, the wireless device 130, and/or the wearable 132 of FIG. 1B.

The resource director 102 further includes, in some examples, the example preference learning module 322. In some examples, based on inputs received in response to the outputs generated by the output generator 314, the preference learning module 322 determines the general preferences of one or more users (e.g., managers, hospital administration, etc.) when accepting/rejecting outputs of the resource director 102. For example, in response to the user consistently taking an action a predetermined percentage (e.g., 95%, 99%, etc.) of given opportunities, the preference learning module 322 can determine, and thereby notify, the resource director 102 that user feedback is not required in making similar decisions in the future.

For example, the preference learning module 322 can determine that the user always accepts schedule substitutions that result in a higher resource composition score and, in response, the resource director 102 no longer queries the user (e.g., manager) regarding substitutions that improve the overall resource composition score. In another example, the preference learning module 322 can determine the user consistently sends home a certain nurse during periods of low census. In such an example, the resource director 102 automatically sends the nurse home during the next period of low census.

In addition to acting automatically without user input as described above, the preference learning module 322 can also receive pre-defined user inputs regarding when it is not necessary to query the user prior to making a change. For example, the user can set the preference learning module 322 to accept all recommended schedules above a pre-determined score threshold.

FIG. 4 is a block diagram of an example implementation of the example score predication engine 312 of FIG. 3. The score prediction engine 312, in some examples, includes at least one of an example neural network 402, an example resource composition generator 404, an example resource composition score engine 406, an example resource composition score accumulator 408, and an example resource composition score storer 410.

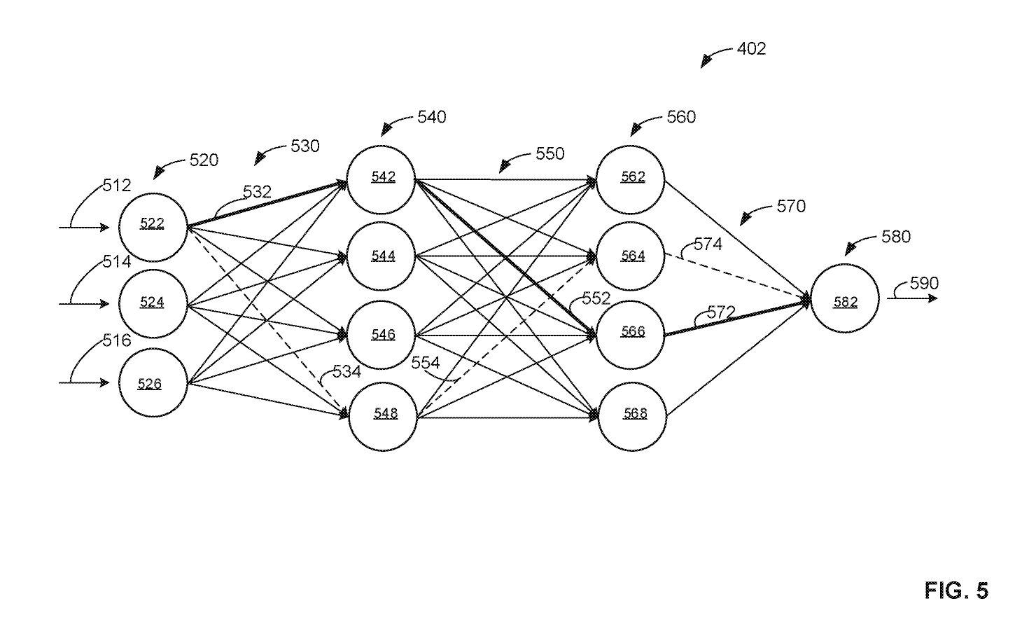

In the example score prediction engine 312 of the example FIG. 4, the resource composition scoring process is driven by the neural network 402, an example data flow of which is further illustrated in FIG. 5, and the resource composition score engine 406. The resource composition score engine 406 utilizes previously acquired outcome data to predict the outcome of potential resource compositions generated by the resource composition generator 404. The neural network 402 is used to train the resource composition score engine 406 to generate more accurate results.

An artificial neural network such as the neural network 402 is a computer system architecture model that learns to do tasks and/or provide responses based on evaluation or “learning” from examples having known inputs and known outputs. A neural network such as the neural network 402 features a series of interconnected nodes referred to as “neurons” or nodes. Input nodes are activated from an outside source/stimulus, such as input from the outcome score storer 306 and the satisfaction score storer 308. The input nodes activate other internal network nodes according to connections between nodes (e.g., governed by machine parameters, prior relationships, etc.). The connections are dynamic and can change based on feedback, training, etc. By changing the connections, an output of the neural network 402 can be improved or optimized to produce more/most accurate results. For example, the neural network 402 can be trained using information from one or more sources to map inputs to potential recommended schedules, etc.

Machine learning techniques, whether neural networks, deep learning networks, and/or other experiential/observational learning system(s), can be used to generate optimal results, locate an object in an image, understand speech and convert speech into text, and improve the relevance of search engine results, for example. Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

For example, deep learning that utilizes a convolutional neural network (CNN) segments data using convolutional filters to locate and identify learned, observable features in the data. Each filter or layer of the CNN architecture transforms the input data to increase the selectivity and invariance of the data. This abstraction of the data allows the machine to focus on the features in the data it is attempting to classify and ignore irrelevant background information.

Deep learning operates on the understanding that many datasets include high level features which include low level features. While examining an image, for example, rather than looking for an object, it is more efficient to look for edges which form motifs which form parts, which form the object being sought. These hierarchies of features can be found in many different forms of data.

Learned observable features include objects and quantifiable regularities learned by the machine during supervised learning. A machine provided with a large set of well classified data is better equipped to distinguish and extract the features pertinent to successful classification of new data.

A deep learning machine that utilizes transfer learning can properly connect data features to certain classifications affirmed by a human expert. Conversely, the same machine can, when informed of an incorrect classification by a human expert, update the parameters for classification. Settings and/or other configuration information, for example, can be guided by learned use of settings and/or other configuration information, and, as a system is used more (e.g., repeatedly and/or by multiple users), a number of variations and/or other possibilities for settings and/or other configuration information can be reduced for a given situation.

An example deep learning neural network can be trained on a set of expert classified data, for example. This set of data builds the first parameters for the neural network, and this would be the stage of supervised learning. During the stage of supervised learning, the neural network can be tested whether the desired behavior has been achieved.

Once a desired neural network behavior has been achieved (e.g., a machine has been trained to operate according to a specified threshold, etc.), the machine can be deployed for use (e.g., testing the machine with “real” data, etc.). During operation, neural network classifications can be confirmed or denied (e.g., by an expert user, expert system, reference database, etc.) to continue to improve neural network behavior. The example neural network is then in a state of transfer learning, as parameters for classification that determine neural network behavior are updated based on ongoing interactions. In certain examples, the neural network such as the neural network 402 can provide direct feedback to another process, such as the resource composition score engine 406, etc. In certain examples, the neural network 402 outputs data that is buffered (e.g., via the cloud, etc.) and validated before it is provided to another process.

In the example of FIG. 4, the neural network 402 receives input from previous outcome data associated with a specific composition of resources stored in the outcome score storer 306 and the satisfaction score storer 308, and outputs an algorithm to predict an outcome score and a satisfaction score associated with other compositions of resources (e.g., a team of doctors and nurses different than the team associated with the scores stored in the outcome score storer 306 and the satisfaction score storer 308). The network 402 can be seeded with some initial correlations and can then learn from ongoing experience. In some examples, the neural network 402 continually receives feedback from at least one of the outcome score storer 306 and the satisfaction score storer 308. Once the neural network 402 reaches a desired level of accuracy (e.g., the network 402 is trained and ready for deployment), the resource composition score engine 406 can be initialized and/or otherwise configured according to a deployed model (e.g., a resource composition score model) of the trained neural network 402. In the example of FIG. 4, throughout the operational life of the score prediction engine 312, the neural network 402 is continuously trained via feedback and the resource composition score engine 406 can be updated based on the neural network 402 and/or outcome score data and satisfaction score data as desired. The network 402 can learn and evolve based on role, location, situation, etc.

In some examples, once the model is deployed to the resource composition engine 406, the resource composition generator 404 can generate one or more possible combinations of resources (e.g., one or more possible staffing schedules) and distribute the possible combinations of resources to the resource composition score engine 406. Utilizing the model, the resource composition score engine 406 outputs one or more predicted outcome scores and satisfaction scores for the received combinations of resources and distributes these to the resource composition score accumulator 408 where the outcomes scores and satisfaction scores are combined to determine one predicted score for each combination of resources. In some examples, the resource composition score accumulator 408 can feedback the highest scoring resource compositions to the resource composition generator 404, which can use this data as seed data to generate improved possible combinations of resources.

In some examples, at least one of the model deployed from the neural network 402 and resource composition scores calculated by the resource composition score engine 406 and the resource composition score accumulator 408 can be stored in the resource composition score storer 410, included in or otherwise implemented by the score prediction engine 312. In some examples, the resource composition score storer 410 can be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). The resource composition score storer 410 can additionally or alternatively be implemented by one or more double data rate (DDR) memories such as DDR, DDR2, DDR3, mobile DDR (mDDR), etc. The resource composition score storer 410 can additionally or alternatively be implemented by one or more mass storage devices such as hard disk drive(s), compact disk drive(s), digital versatile disk drive(s), etc. While in the illustrated example the resource composition score storer 410 is illustrated as a single database, the resource composition score storer 410 can be implemented by any number and/or type(s) of databases. Further, the data stored in the resource composition score storer 410 can be in any format such as binary data, comma delimited data, tab delimited data, structured query language (SQL) structures, etc.

FIG. 5 is an example representation of the example neural network 402 of FIG. 4. The example neural network 402 includes layers 520, 540, 560, and 580. The layers 520 and 540 are connected with neural connections 530. The layers 540 and 560 are connected with neural connections 550. The layers 560 and 580 are connected with neural connections 570. Data flows forward via inputs 512, 514, 516 from the input layer 520 to the output layer 580 and to an output 590.

The layer 520 is an input layer that, in the example of FIG. 5, includes a plurality of nodes 522, 524, 526. The layers 540 and 560 are hidden layers and include, in the example of FIG. 5, nodes 542, 544, 546, 548, 562, 564, 566, 568. The neural network 402 can include more or less hidden layers 540 and 560 than shown. The layer 580 is an output layer and includes, in the example of FIG. 5, a node 582 with an output 590. Each input 512-516 corresponds to a node 522-526 of the input layer 520, and each node 522-526 of the input layer 520 has a connection 530 to each node 542-548 of the hidden layer 540. Each node 542-548 of the hidden layer 540 has a connection 550 to each node 562-568 of the hidden layer 560. Each node 562-568 of the hidden layer 560 has a connection 570 to the output layer 580. The output layer 580 has an output 590 to provide an output from the example neural network 402.

Of connections 530, 550, and 570 certain example connections 532, 552, 572 can be given added weight while other example connections 534, 554, 574 can be given less weight in the neural network 402. Input nodes 522-526 are activated through receipt of input data via inputs 512-516, for example. Nodes 542-548 and 562-568 of hidden layers 540 and 560 are activated through the forward flow of data through the network 402 via the connections 530 and 550, respectively. Node 582 of the output layer 580 is activated after data processed in hidden layers 540 and 560 is sent via connections 570. When the output node 582 of the output layer 580 is activated, the node 582 outputs an appropriate value based on processing accomplished in hidden layers 540 and 560 of the neural network 402.

For example, the inputs 512, 514, and 516 to the neural network 402 can include at least one of outcome scores and satisfaction scores for one or more previous resource compositions that flows into the input layer 520. From the input layer 520, the scores for the previous resource compositions are distributed to the hidden layer 540 (e.g., the layer is hidden as the outputs of the layer are not displayed in a user facing manner) via the connections 530. In some examples, at the hidden layer 540, the nodes 542-548 can each consider different permutations of the resource compositions received via the connections 530. Further in such examples, the best performing permutations from the nodes 542-548 are further distributed to the hidden layer 560 via the connections 550 and the nodes 562-568 further permutate the best performing permutations. The nodes 562-568 calculate scores for the permutated resource compositions and distribute the scores to the output node 582. Once the output node 582 is activated, the output node 582 can determine a scoring model based on the permutated resource compositions and their corresponding scores received via the connections 570. Further, the output node 582 can distribute an output 590, wherein the output 590 can include the model including one or more correlation coefficients, the coefficients correlating scores to one or more resources and/or combination of resources.

FIG. 6 is a block diagram of an implementation of the example schedule optimizer 316 of FIG. 3. The schedule optimizer 316, in some examples, includes at least one of an example constraint applicator 602, an example schedule generator 604, an example schedule selector 606, and an example schedule storer 608.

The constraint applicator 602, included in or otherwise implemented by the output generator 314, applies each of the known schedule constraints received from the scheduling constraint storer 304 to the resource compositions received from the score prediction engine 312. In some examples, the constraint applicator 602 may only apply the hard constraints received from the scheduling constraint storer 304. Additionally or alternatively, the constraint applicator 602 can apply both the hard constraints and the soft constraints received from the scheduling constraint storer 304.

The schedule generator 604, included in or otherwise implemented by the output generator 314, can generate a plurality of possible schedules based on the constraints applied by the constraint applicator 602 to the plurality of resource compositions. In some examples, the schedule generator 604 is an exhaustive generator that attempts to generate a majority of all possible schedules.

The schedule selector 606, included in or otherwise implemented by the output generator 314, selects a portion of the schedules generated by the schedule generator 604 to be distributed to a user of the system 100, via the communications manager 302, and to the schedule storer 608. In some examples, the portion of the schedules to be distributed is based on the predicted scores of the schedules as determined by the score prediction engine 312. For example, the top 3 schedules, ranked by score, can be distributed for viewing by the user and for storage. Additionally or alternatively, the schedule selector 606 can select a schedule based on the predicted scores and one or more preferences as determined by the preference learning module 322. For example, if one combination of resources marginally decreases the predicted score but has been consistently preferred by the user of the system 100, the schedule selector 606 can output the schedule including the typical user preference.

Additionally or alternatively, the schedule selector 606 can select one or more schedules to output based on only one of predicted outcome scores or satisfaction scores. Additionally, the schedule selector 606 can select one or more schedules to output based on an uneven weight of predicted outcome scores and satisfaction scores (e.g., outcomes weighted more, satisfaction weighted more, etc.). Additionally or alternatively, the schedule selector 606 can select one or more schedules to output based on a pre-defined preference of the user as distributed by the preference learning module 322. For example, the pre-defined preference of the user can be to prefer cost-saving schedules and the schedule selector 606 can, in response, output the highest performing schedule(s) that adhere to a pre-defined cost threshold.

The schedule storer 608, included in or otherwise implemented by the output generator 314, can store the one or more schedules generated by the schedule selector 606 and/or the deployed schedule as selected by the user. In some examples, the schedule storer 608 can be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). The schedule storer 608 can additionally or alternatively be implemented by one or more double data rate (DDR) memories such as DDR, DDR2, DDR3, mobile DDR (mDDR), etc. The schedule storer 608 can additionally or alternatively be implemented by one or more mass storage devices such as hard disk drive(s), compact disk drive(s), digital versatile disk drive(s), etc. While in the illustrated example the schedule storer 608 is illustrated as a single database, the schedule storer 608 can be implemented by any number and/or type(s) of databases. Further, the data stored in the schedule storer 608 can be in any format such as binary data, comma delimited data, tab delimited data, structured query language (SQL) structures, etc.

In one operational example of the schedule optimizer 316, Nurse Manager Susan determines that she needs to generate a schedule for her unit. Per hospital regulations, the generated schedule needs to encompass the next 6-weeks. This task typically takes her 6-8 hours and she often does not feel confident in one or more of the resource compositions included in the schedule as she has a limited quantity of time to consider all of the resource compositions that would be possible within the confines of the scheduling constraints provided to her. In such examples, Nurse Manager Susan can utilize the resource director 102 to instead generate multiple schedules that are possible within the confines of the scheduling constraints and include resource compositions with the highest predicted outcome scores and satisfaction scores.

For example, Nurse Manager Susan can initialize this routine with an input to a computing device (e.g., a wearable, a wireless device, a computing terminal, etc.) which is received by the resource director 102. The resource director 102, in response to receiving the request, generates one or more resource composition scores with the score prediction engine 312 which are sent to the schedule optimizer 316. The scheduling optimizer 316 then applies scheduling constraints to the received resource compositions with the constraint applicator 602, generates one or more schedules satisfying the constraints with the schedule generator 604, and selects one or more of the generated schedules to present to Nurse Manager Susan based on predicted scores of the resource compositions included in the schedule generated by the score prediction engine 312. In some examples, each of the aforementioned actions are performed in real time (or substantially real time given data retrieval, processing, and transmission latency), and Nurse Manager Susan receives the selected schedules in the same computing session in which she requested the schedules.

Nurse Manager Susan can now select from one of the presented schedules using, for example, the scheduling recommendation system 129 displayed on the computing terminal 128. Additionally or alternatively, Nurse Manager Susan may like one of the presented schedules (e.g., a second presented schedule), but disagrees with one decision the resource director 102 made for the fourth day of the schedule (e.g., too many nurses scheduled). In such an example, Nurse Manager Susan can use the computing terminal 128 to modify the schedule, removing one of the scheduled nurses in the described example, and accept the schedule after completing the modification. Upon completion of accepting the schedule, Nurse Manager Susan can return to other tasks. While Nurse Manager Susan and her nursing team are described in the above example, the first operational example of the schedule optimizer 316 can be used for any team and/or department at the hospital. For example, Janitorial Staff Manager Noah can generate and/or modify a schedule for the Janitorial team utilizing the resource director 102.

In a second operational example of the schedule optimizer 316, Nurse Manager Susan again determines that she needs to generate a schedule for her unit. Per hospital regulations, the generated schedule needs to encompass the next 6-weeks. However, in this example, Nurse Manager Susan prefers to manually generate schedules, but she still does not have time to consider all of the resource compositions that would be possible within the confines of the scheduling constraints provided to her. In such examples, Nurse Manager Susan can utilize the resource director 102 to monitor her selections. In some examples, monitoring her selections can include utilizing the schedule optimizer 316 to notify Nurse Manger Susan when a resource composition exists that is associated with a higher prediction score (e.g., at least one of a higher outcome score and/or a higher satisfaction score) than a resource composition she had manually selected. In such an example, Nurse Manager Susan can accept or reject the modification suggested by the resource director 102 via the scheduling recommendation system 129 displayed on the computing terminal 128. Utilizing the resource director 102 in such a manner, Nurse Manager Susan can be confident that the resource compositions that she selected and/or were recommended by the resource director 102 can lead to positive patient outcomes and high satisfaction scores for employees and/or patients.

FIG. 7 is a block diagram of an example implementation of the example substitution recommender 318 of FIG. 3. The substitution recommender 318, in some examples, includes at least one of an example event processor 702, an example resource substitution generator 704, an example substitution selector 706, and an example substitution storer 708.

The event processor 702, included in or otherwise implemented by the score prediction engine 312, processes a scheduling event received from the scheduling event storer 310. In some examples, processing the scheduling event further includes determining a type of scheduling event (e.g., low census, high census, trade request, etc.) in addition to a severity of a scheduling event (e.g., the magnitude of low/high census, importance of trade request, etc.). In some examples, the event processor 702 is further to distribute characteristics of the scheduling event to the substitution generator 704.

The substitution generator 704, included in or otherwise implemented by the substitution recommender 318, determines a plurality of scheduling substitutions that satisfy the scheduling constraints received from the scheduling constraint storer 304 and remedy the characteristics of the scheduling event received from the event processor 702. For example, the substitution generator 704 can select a quantity of employees (e.g., nurses, doctors, etc.) to call in based on the magnitude of a high census event. Additionally or alternatively, the substitution generator 704 can select a quantity of employees to send home based on the magnitude of a low census event. Additionally or alternatively, the substitution generator 704 can determine one or more employees capable of fulfilling a trade request with the requesting employee. In each case, each employee identified, along with the characteristics of the scheduling event, are distributed to the substitution selector 706.

The substitution selector 706, included in or otherwise implemented by the substitution recommender 318, determines a portion of the employees identified (e.g., the employees identified to be called in, the employees identified to be sent home, the employees identified as potential trades, etc.) to be distributed to the user of the system 100 via the communications manager 302. For example, based on data received from the score prediction engine 312, the substitution selector 706, in response to a low census event, can select one or more employees with the lowest predicted scores as determined by the score prediction engine 312 to be sent home. Additionally or alternatively, in response to a high census event, the substitution selector 706 can select one or more employees with the highest predicted scores as determined by the score prediction engine 312 to be called in. Additionally or alternatively, in response to a trade request, the substitution selector 706 can select one or more employees with the best possible impact on the overall predicted resource composition score (e.g., the impact of the trade would impact the resource composition positively or least negatively) to be possible trade partners. In addition to using the predicted scores received from the score prediction engine 312, the substitution selector 706 can, in some examples, utilize other known management criteria (e.g., minimizing financial impact, etc.) when determining the portion of the employees identified by the substitution selector 706 to be distributed to the communication manager 302.

The substitution storer 708, included in or otherwise implemented by the substitution recommender 318, can store the portion of employees identified by the substitution selector 706. Additionally or alternatively, in some examples, the substitution storer 708 can store a user selection of one or more of the employees of the portion of employees. In some examples, the substitution storer 708 can be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). The substitution storer 708 can additionally or alternatively be implemented by one or more double data rate (DDR) memories such as DDR, DDR2, DDR3, mobile DDR (mDDR), etc. The substitution storer 708 can additionally or alternatively be implemented by one or more mass storage devices such as hard disk drive(s), compact disk drive(s), digital versatile disk drive(s), etc. While in the illustrated example the substitution storer 708 is illustrated as a single database, the substitution storer 708 can be implemented by any number and/or type(s) of databases. Further, the data stored in the substitution storer 708 can be in any format such as binary data, comma delimited data, tab delimited data, structured query language (SQL) structures, etc.

In operational examples of the substitution recommender 318, Nurse Manager Tammy, in charge of the 3-West wing of a hospital, must routinely fill in scheduling gaps in her unit. This task, while not typically requiring a significant expenditure of time, must be done dynamically and on the fly, and Tammy often does not feel confident in the resources she selected to fill the scheduling gaps as she has a limited quantity of time to consider all of the resources that would be possible to fill the gaps within the confines of the scheduling constraints provided to her. In such examples, Nurse Manager Tammy can utilize the resource director 102 to instead dynamically generate multiple resource substitutions that are possible within the confines of the scheduling constraints and result in resource compositions with the highest predicted outcome scores and satisfaction scores.

For example, Nurse Manager Tammy, in some examples, is not required to initialize this process. Instead, the resource director 102 can automatically determine the presence of a scheduling event and automatically begin the process flow in response. The resource director 102, in response to receiving the scheduling event (e.g., a low census event, a high census event, a shift trade request, etc.), generates one or more resource composition scores with the score prediction engine 312 which are sent to the substitution recommender 318. The substitution recommender 318 then determines the severity of the staffing decision with the event processor 702, generates one or more resource substitutions satisfying the constraints with the substitution generator 704, and selects one or more of the substitutions to present to Nurse Manager Tammy with the substitution selector 706 based on predicted scores of the resource compositions created by the substitutions by the score prediction engine 312. In some examples, each of the aforementioned actions are performed dynamically, and Nurse Manager Tammy receives the recommended substitutions in real time (or substantially real time given data retrieval, processing, and transmission latency) compared to when the scheduling event was received by the resource director 102.

Nurse Manager Tammy can now select from one of the presented substitutions using, for example, the wearable 132.

In one operational example of the substitution recommender 318, the resource director 102 receives a high census event from the server 120 or a call-in (e.g., Nurse Michelle calls in sick for her shift occurring that day) from the server 121. In such an example, the resource director 102 presents one or more resources to call in. For example, the resource director 102 can present a recommendation to call in Nurse Aaron because his predicted score on the team is higher than Nurse Tim. In other examples, the resource director 102 can present a recommendation to call in both Nurse Aaron and Nurse Tim because two resources are needed. In yet other examples, the resource director 102 can present a recommendation to try to call in Nurse Aaron first, Nurse Tim second and Nurse Joy, whose predicted score is lower than both Aaron and Tim, third. In such an example, Nurse Manager Tammy can accept or reject any one of the recommendations suggested by the resource director 102 on the display of the wearable 132, for example. In response to Nurse Tammy making a selection, the selection is distributed to the server 126, where it is propagated to any employee effected by the substitution.

In a second operational example of the substitution recommender 318, the resource director 102 receives a low census event from the server 120. In such an example, the resource director 102 presents one or more resources to call off. For example, the resource director 102 can present a recommendation to call off Nurse Joy because her predicted score on the team is lower than Nurse Bill, who is also working the same shift. In other examples, the resource director 102 can present a recommendation to call off both Nurse Joy and Nurse Bill because the shift requires two less resources than are currently scheduled. In such an example, Nurse Manager Tammy can accept or reject any one of the recommendations suggested by the resource director 102 on the display of the wearable 132, for example. In response to Nurse Tammy making a selection, the selection is distributed to the server 126, where it is propagated to any employee effected by the substitution.

In a third operational example of the substitution recommender 318, the resource director 102 receives a shift trade request (e.g., the shift trade is requested by Nurse Florence) from the computational terminal 122. In such an example, the resource director 102 presents one or more resources to trade with Nurse Florence. For example, the resource director 102 can present a recommendation to trade Nurse Florence's shift with one of Nurse Karlene's shift or Nurse Joy's shift the following day, as either shift trade improves the predicted score of each of their respective teams. In some examples, the resource director 102 can further present a more detailed analysis noting that a trade with Nurse Karlene would have a more significant effect on outcome scores whereas a trade with Nurse Joy would have a significant effect on satisfaction scores. In such examples, Nurse Manager Tammy can accept or reject any one of the recommendations suggested by the resource director 102 on the display of the wearable 132, for example. In some examples, Nurse Manager Tammy can have a preference for satisfaction scores and selects the trade with Nurse Joy. Alternatively, Nurse Manger Tammy can have a preference for outcome scores and selects Nurse Karlene. In some examples, this preference can be stored in the server 126. In response to Nurse Tammy making a selection, the selection is distributed to the server 126, where it is propagated to any employee effected by the substitution.

In a fourth operational example of the substitution recommender 318, the resource director 102 receives a staffing imbalance (e.g., a Post Op unit is overstaffed and a ICU unit is understaffed, etc.). In such an example, the resource director 102 presents one or more resources to shift from the Post Op Unit to the ICU unit. For example, the resource director 102 can present a recommendation to shift Nurse Joy from the Post Op unit to the ICU unit as it has the greatest effect on the predicted score for the ICU unit. Additionally, the resource director 102 can present a recommendation to shift Nurse Jim from the Post Op unit to the ICU unit as it has the least negative effect on the predicted score of the Post Op unit. In such examples, Nurse Manager Tammy can accept or reject any one of the recommendations suggested by the resource director 102 on the display of the wearable 132, for example. In some examples, Nurse Manager Tammy can have a preference for the receiving unit and selects Nurse Joy. Alternatively, Nurse Manger Tammy can have a preference for the shifting unit and selects Nurse Jim. In some examples, this preference can be stored in the server 126. In response to Nurse Tammy making a selection, the selection is distributed to the server 126, where it is propagated to any employee effected by the substitution.

FIG. 8 is a block diagram of an example implementation of the example resource manager 320 of FIG. 3. The resource manager 320, in some examples, includes at least one of an example resource score sorter 802, an example ranked resource generator 804, an example commendation recommender 806, and a ranked resource storer 808.

The resource score sorter 802, included in or otherwise implemented by the resource manager 320, can utilize the predicted resource composition scores received from the score prediction engine 312 to sort a plurality of resources by score. For example, the resource score sorter 802 can sort the resources by overall score. Additionally or alternatively, the resource score sorter 802 can sort the resources by outcome score. Additionally or alternatively, the resource score sorter 802 can sort the resources by satisfaction score. In response to completing the sorting process, the resource score sorter 802 is further to distribute the sorted list of resources to the ranked resource generator 804.

The ranked resource generator 804, included in or otherwise implemented by the resource manager 320, ranks the list of resources received from the resource score sorter 802 in descending order of score (e.g., from a high score to a low score), the score defined by at least one of the overall scores, the outcomes scores, and/or the satisfaction scores. In some examples, the ranked resource generator 804, in addition to generating the ranking of the resources, is further to generate one or more pools (e.g., strata) of resources based on their scores. For example, the ranked resource generator 804 can take the top strata of resources (e.g., the top 10%, top 25%, etc.) and generate a top performing pool (e.g., a float pool). Additionally or alternatively, the ranked resource generator 804 can take the bottom strata of resources (e.g., the bottom 10%, bottom 25%, etc.) and generate a low performing pool. In response to generating at least of one of the ranked order of resources, the top performing pool, and the low performing pool, the ranked resource generator can distribute at least one of these data sets to at least one of the communications manager 302, the commendation recommender 806, and the ranked resource storer 808.

The commendation recommender 806, included in or otherwise implemented by the resource manager 320, can determine a commendation (e.g., financial compensation, a physical or virtual reward, etc.) to be provided to one or more resources based on at least one of their ranking or their presence in at least one of the top performing pool and/or the low performing pool. For example, the commendation recommender 806 can recommend financial compensation to scale as a function of the ranking of resources. Additionally or alternatively, the commendation recommender 806 can recommend a certain reward, either virtual or physical, to be awarded to one or more resources in the top performing pool.

The ranked resource storer 808, included in or otherwise implemented by the resource manager 320, can store at least one of the ranking of resources, the top performing pool of resources, and the low performing pool of resources received from the ranked resource generator 804. In some examples, the ranked resource storer 808 can be implemented by a volatile memory (e.g., a Synchronous Dynamic Random Access Memory (SDRAM), Dynamic Random Access Memory (DRAM), RAMBUS Dynamic Random Access Memory (RDRAM), etc.) and/or a non-volatile memory (e.g., flash memory). The ranked resource storer 808 can additionally or alternatively be implemented by one or more double data rate (DDR) memories such as DDR, DDR2, DDR3, mobile DDR (mDDR), etc. The resource composition score storer 410 can additionally or alternatively be implemented by one or more mass storage devices such as hard disk drive(s), compact disk drive(s), digital versatile disk drive(s), etc. While in the illustrated example the ranked resource storer 808 is illustrated as a single database, the ranked resource storer 808 can be implemented by any number and/or type(s) of databases. Further, the data stored in the ranked resource storer 808 can be in any format such as binary data, comma delimited data, tab delimited data, structured query language (SQL) structures, etc.

While an example implementation of the resource director 102 of FIGS. 1A and 1B is illustrated in FIGS. 3-8, one or more of the elements, processes, and/or devices illustrated in FIGS. 3-8 can be combined, divided, re-arranged, omitted, eliminated, and/or implemented in any other way. Further, the example communication manager 302, the example scheduling constraint storer 304, the example outcome score storer 306, the example satisfaction score storer 308, the example scheduling event storer 310, the example score predication engine 312, the example output generator 314, the example schedule optimizer 316, the example substitution recommender 318, the example resource manager 320, the example preference learning module 322, and/or, more generally, the example resource director 102 of FIGS. 1A and 1B can be implemented by hardware, software, firmware, and/or any combination of hardware, software, and/or firmware. Thus, for example, any of the example communication manager 302, the example scheduling constraint storer 304, the example outcome score storer 306, the example satisfaction score storer 308, the example scheduling event storer 310, the example score predication engine 312, the example output generator 314, the example schedule optimizer 316, the example substitution recommender 318, the example resource manager 320, the example preference learning module 322, and/or, more generally, the example resource director 102 of FIGS. 1A and 1B can be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)), and/or field programmable logic device(s) (FPLD(s)). When reading any of the apparatus or system claims of this patent to cover a purely software and/or firmware implementation, at least one of the example communication manager 302, the example scheduling constraint storer 304, the example outcome score storer 306, the example satisfaction score storer 308, the example scheduling event storer 310, the example score predication engine 312, the example output generator 314, the example schedule optimizer 316, the example substitution recommender 318, the example resource manager 320, and/or the example preference learning module 322 is/are hereby expressly defined to include a non-transitory computer readable storage device or storage disk such as a memory, a digital versatile disk (DVD), a compact disk (CD), a Blu-ray disk, etc., including the software and/or firmware. Further still, the example resource director 102 of FIGS. 1A and 1B can include one or more elements, processes, and/or devices in addition to, or instead of, those illustrated in FIGS. 3-8, and/or can include more than one of any or all of the illustrated elements, processes, and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

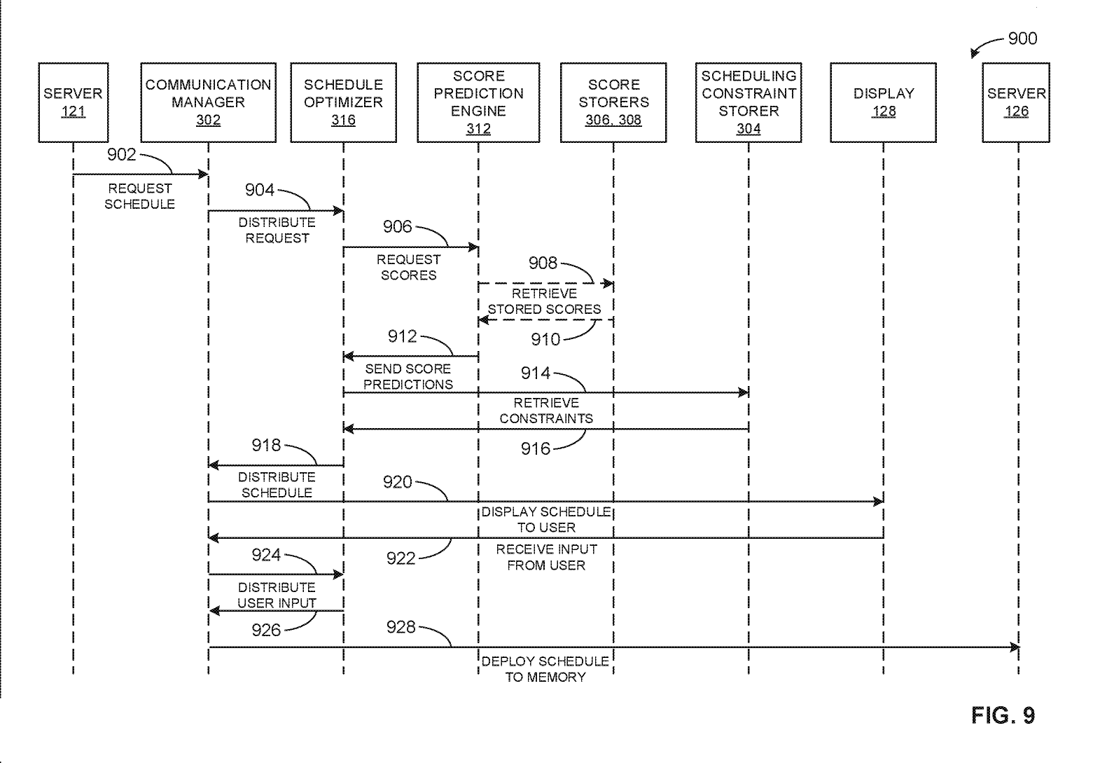

FIG. 9 illustrates a first example data flow and transformation of information 900 as it flows among the components of the system 100. As shown in the example of FIG. 9, a schedule request 902 is provided by the server 121 to the communication manager 302. For example, the server 121 can distribute the schedule request 902 in response to determining that a schedule has not been previously generated for an upcoming time period. Additionally or alternatively, while not illustrated in FIG. 9, a user can initialize the schedule request 902 from a device such as the wearable 104, the wireless device 108, and/or the computing terminal 122 of FIG. 1A. The communication manager 302 transforms the schedule request 902 into a schedule request 904, wherein the schedule request 904 is readable by the schedule optimizer 316. In response to receiving the schedule request 904, the schedule optimizer 316 is further to generate a score request 906 and distribute the score request 906 to the score prediction engine 312. In response to receiving the score request 906, the score prediction engine 312 is further to distribute a stored data request 908 to at least one of the outcome score storer 306 and the satisfaction score storer 308, which are to return the requested scores (the requested score including at least one of outcome scores and satisfaction scores associated with prior sets of resource compositions) to the score prediction engine 312 as training data 910.

Utilizing the training data 910, the score prediction engine 312 predicts resource composition scores for one more combinations (e.g., compositions) of resources for which no score data is present (e.g., the composition of resources has not previously occurred) and distributes the resource composition scores to the schedule optimizer 316 as predicted scores transmittal 912. In response to receiving the predicted scores transmittal 912, the schedule optimizer 316 queries the scheduling constraint storer 304 with a constraint request 914, the constraint request 914 to request one or more constraints for the time period of the resource schedule requested by the server 121, and the scheduling constraint storer 304 returns the requested constraints to the schedule optimizer 316 as schedule constraint transmittal 916. In response to receiving the schedule constraint transmittal 916, the schedule optimizer 316 applies the schedule constraint transmittal 916 to the predicted scores 912 to determine one or more recommended schedules for the time period requested by the server 121 and distributes the one or more recommended schedules to the communication manager 302 as recommended schedule transmittal 918.

In response to receiving the recommended schedule transmittal 918, the communication manager 302 distributes the recommended schedule transmittal 918 to the computing terminal 128 as recommended schedule transmittal 920. A user of the computing terminal 128 receives the recommended schedule transmittal 920 and, utilizing an interface of the computing terminal 128, accepts or modifies one of the recommended schedules included with the recommended schedule transmittal 920. In both cases, the input of the user is distributed to the schedule optimizer 316 via the communication manager 302 with a user input transmittal 922 and 924. In response to receiving the user input with the input transmittal 924, the schedule optimizer 316 applies the user modification to one of the recommended schedules or applies the recommended schedule. The schedule optimizer 316 then returns the selected schedule, the selected schedule corresponding to the time period requested previously by the server 121, to the server 126 via the communication manager 302 for deployment to the server 126 with a selected schedule transmittal 926 and 928.

Thus, certain examples transform patient information, patient outcome data, patient satisfaction data, and employee satisfaction data into resource scheduling information such as a resource schedule calculated to match a set of pre-determined or dynamic constraints and/or goals that results in the reconfiguration of one or more properties of the server 126. For example, as described above, using machine learning, such as the neural network 402, etc., a plurality of parameters, settings, etc., can be developed, monitored, and refined through deployment of various resource schedules and combinations of resources, for example. Using the neural network 402, for example, learning/training and testing can be facilitated before the resource scheduling system is deployed (e.g., in an internal or testing environment using previously acquired outcome score and satisfaction score data), while continued adjustment of parameters occurs “in the field” (e.g., using outcome score and satisfaction score data for schedules and/or resource compositions generated by the neural network 402) after the system has been deployed and activated for use, for example.

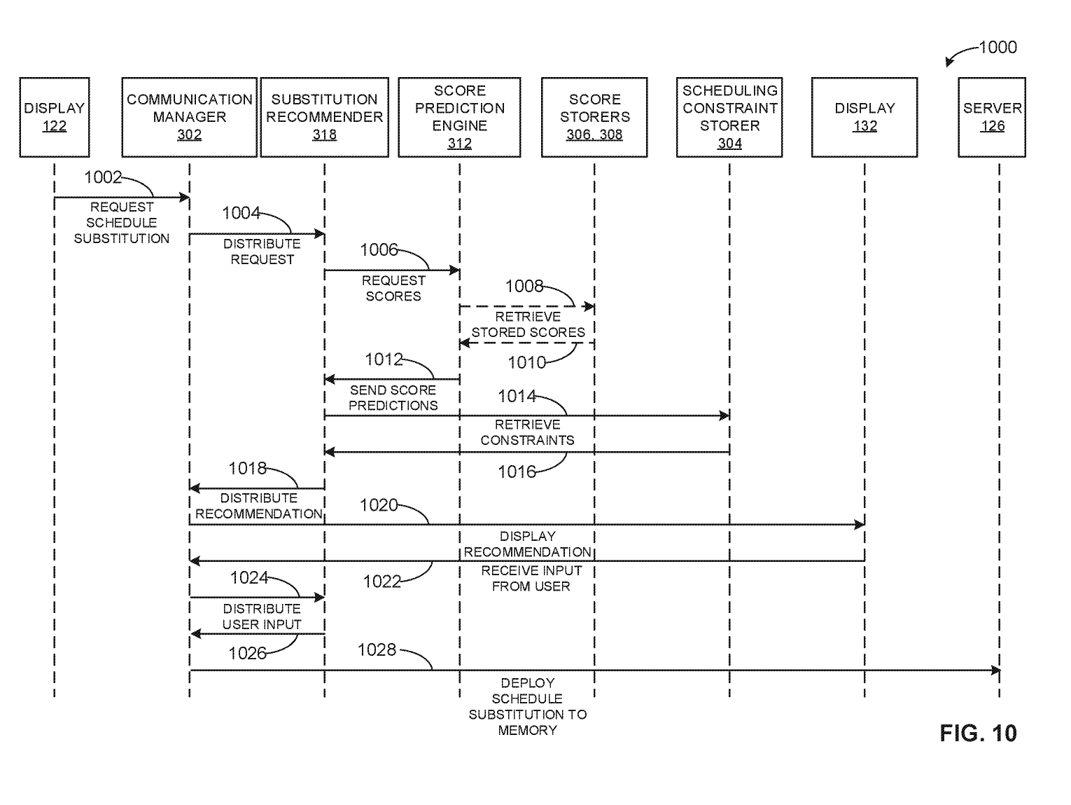

FIG. 10 illustrates a second example data flow and transformation of information 1000 as it flows among the components of the system 100. As shown in the example of FIG. 10, a substitution request 1002 is provided by the computing terminal 122 to the communication manager 302. For example, the substitution request 1002 can be initialized in response to a user input (e.g., a scheduling request) to the scheduling request system 123 displayed on the computing terminal 122. Additionally or alternatively, while not illustrated in FIG. 10, the substitution request 1002 can be initialized in response to at least one of a low census event or high census event received from the server 120. The communication manager 302 transforms the substitution request 1002 into a substitution request 1004, wherein the substitution request 1004 is readable by the substitution recommender 318. In response to receiving the substitution request 1004, the substitution recommender 318 is further to generate a score request 1006 and distribute the score request 1006 to the score prediction engine 312. In response to receiving the score request 1006, the score prediction engine 312 is further to distribute stored data request 1008 (e.g., a request for previously stored scores) to at least one of the outcome score storer 306 and the satisfaction score storer 308 which are to return the requested scores (the requested score including at least one of outcome scores and satisfaction scores associated with prior sets of resource compositions) to the score prediction engine 312 as training data 910.

Utilizing the training data 1010, the score prediction engine 312 predicts resource composition scores for one more combinations (e.g., compositions) of resources for which no score data is present (e.g., the composition of resources has not previously occurred) and distributes the resource composition scores to the schedule optimizer 316 as predicted scores transmittal 1012. In response to receiving the predicted scores transmittal 1012, the substitution recommender 318 queries the scheduling constraint storer 304 with a constraint request 1014, the constraint request 1014 to request one or more constraints for the time period of the substitution requested by the computing terminal 122, and the scheduling constraint storer 304 returns the requested constraints to the schedule optimizer 316 as constraint transmittal 1016. In response to receiving the constraint transmittal 1016, the substitution recommender 318 applies the constraints received from constraint transmittal 1016 to the score predictions received from predicted scores transmittal 1012 to determine one or more recommended schedule substitutions (e.g., one or more schedule trades, one or more resources to send home during low census, one or more resources to call in during high census, etc.) and distributes the one or more recommended schedule substitutions to the communication manager 302 as recommended substitution transmittal 1018.

In response to receiving the recommended substitution transmittal 1018, the communication manager 302 distributes the one more recommended schedule substitutions included with the recommended substitution transmittal 1018 to the wearable 132, for example, as recommended substitution transmittal 1020. A user of the wearable 132 receives the recommended substitution transmittal 1020 and, utilizing an interface of the wearable 132, accepts one of the recommended schedule substitutions or modifies one of the recommended schedule substitutions included in the recommended substitution transmittal 1020. In both cases, the input of the user is distributed to the substitution recommender 318 via the communication manager 302 with a user input transmittal 1022 and 1024. In response to receiving the user input with the input transmittal 1024, the substitution recommender 318 applies the user modification to one of the recommended schedule substitutions or applies the user selected schedule substitutions. The substitution recommender 318 then distributes the schedule substitution to the server 126 via the communication manager 302, the schedule substitution to modify an existing resource schedule in the server 126 with a selected substitution transmittal 1026 and 1028.

Thus, certain examples transform patient information, patient outcome data, patient satisfaction data, employee satisfaction data, and a schedule substitution request into resource scheduling information such as a recommended schedule substitution calculated to match a set of pre-determined and/or dynamic constraints and/or goals that results in the reconfiguration of one or more properties of the server 126. For example, as described above, the system 100 can transform at least one of a notification of a schedule trade request, a low census event, and/or a high census event into a recommended schedule modification (e.g., a recommended shift trade, a recommended resource to send home or call in, etc.) Using machine learning, such as the neural network 402, etc., a plurality of parameters, settings, etc., can be developed, monitored, and refined through deployment of various resource schedules and combinations of resources, for example. Using the neural network 402, for example, learning/training and testing can be facilitated before the resource scheduling system is deployed (e.g., in an internal or testing environment using previously acquired outcome score and satisfaction score data), while continued adjustment of parameters occurs “in the field” (e.g., using outcome score and satisfaction score data for schedules and/or resource compositions generated by the neural network 402) after the system has been deployed and activated for use, for example.

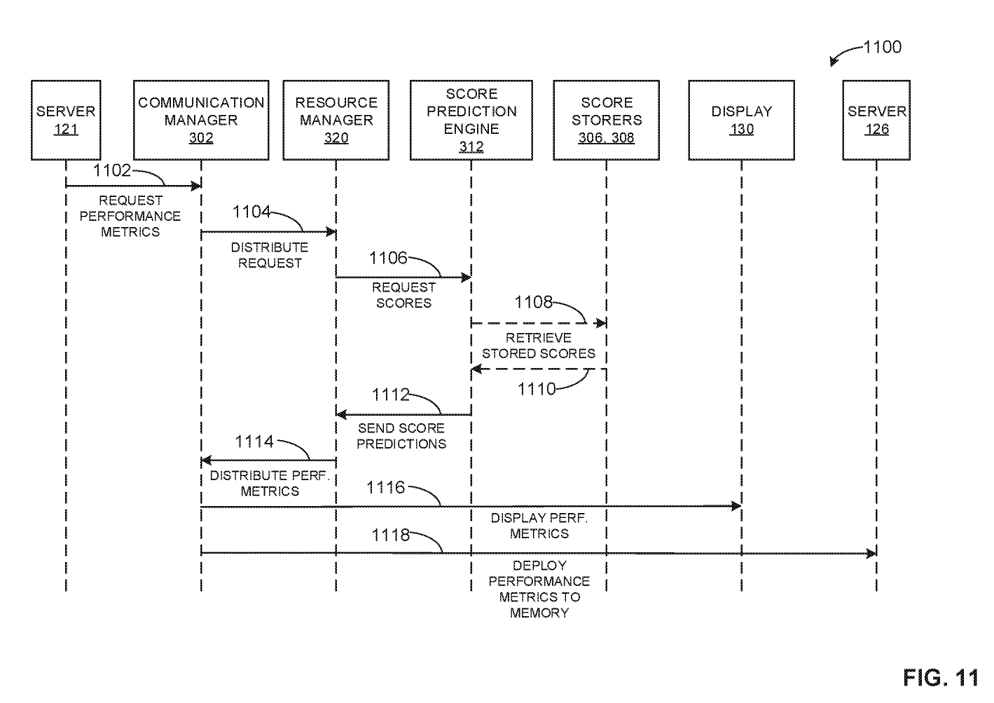

FIG. 11 illustrates a third example data flow and transformation of information 1100 as it flows among the components of the system 100. As shown in the example of FIG. 1, a metrics request 1102 (e.g., a performance metrics request) is provided by the server 121 to the communication manager 302. Additionally or alternatively, while not illustrated in FIG. 11, the metrics request 1102 can be initialized in response to a user input to at least one of the wearable 104, the wireless device 108, and/or the computing terminal 122 as illustrated in FIG. 1A. The communication manager 302 transforms the metrics request 1102 into a metrics request 1104, wherein the metrics request 1104 is readable by the resource manager 320. In response to receiving the metrics request 1104, the resource manager 320 is further to generate a score request 1106 and distribute the score request 1106 to the score prediction engine 312. In response to receiving the score request 1106, the score prediction engine 312 is further to distribute a stored data request 1108 (e.g., a request for previously stored scores) to at least one of the outcome score storer 306 and the satisfaction score storer 308 which are to return the requested scores (the requested score including at least one of outcome scores and satisfaction scores associated with prior sets of resource compositions) to the score prediction engine 312 as training data 1110.

Utilizing the training data 1110, the score prediction engine 312 predicts resource composition scores for one more combinations (e.g., compositions) of resources for which no score data is present (e.g., the composition of resources has not previously occurred) and distributes the resource composition scores to the resource manager 320 as predicted scores transmittal 1112. In response to receiving the predicted scores transmittal 1112, the resource manager 320 determines one or more top performing resources (e.g., one or more resources with highest prediction score) and organizes the top performing resources as a “float pool” (e.g., a pool of resources to substitute and/or shift around based on need) based on the resource composition scores included with the predicted scores transmittal 1112. The resource manager 320 distributes the float pool to the communication manager 302 as a pool transmittal 1114. The communication manager 302 then distributes the float pool to at least one of the wireless device 130 for display to a user and the server 126 for storage and/or modification of a previous float pool stored in the server 126 via pool transmittal 1116 and pool transmittal 1118, respectively.

Thus, certain examples transform patient information, patient outcome data, patient satisfaction data, and employee satisfaction data into resource scheduling information such as a resource float pool calculated to match a set of pre-determined or dynamic constraints and/or goals that results in the reconfiguration of one or more properties of the server 126. For example, as described above, the system 100 can transform a request to update a pool of resources into a modification of the pool of resources. Using machine learning, such as the neural network 402, etc., a plurality of parameters, settings, etc., can be developed, monitored, and refined through deployment of various resource schedules and combinations of resources, for example. Using the neural network 402, for example, learning/training and testing can be facilitated before the resource scheduling system is deployed (e.g., in an internal or testing environment using previously acquired outcome score and satisfaction score data), while continued adjustment of parameters occurs “in the field” (e.g., using outcome score and satisfaction score data for schedules and/or resource compositions generated by the neural network 402) after the system has been deployed and activated for use, for example.

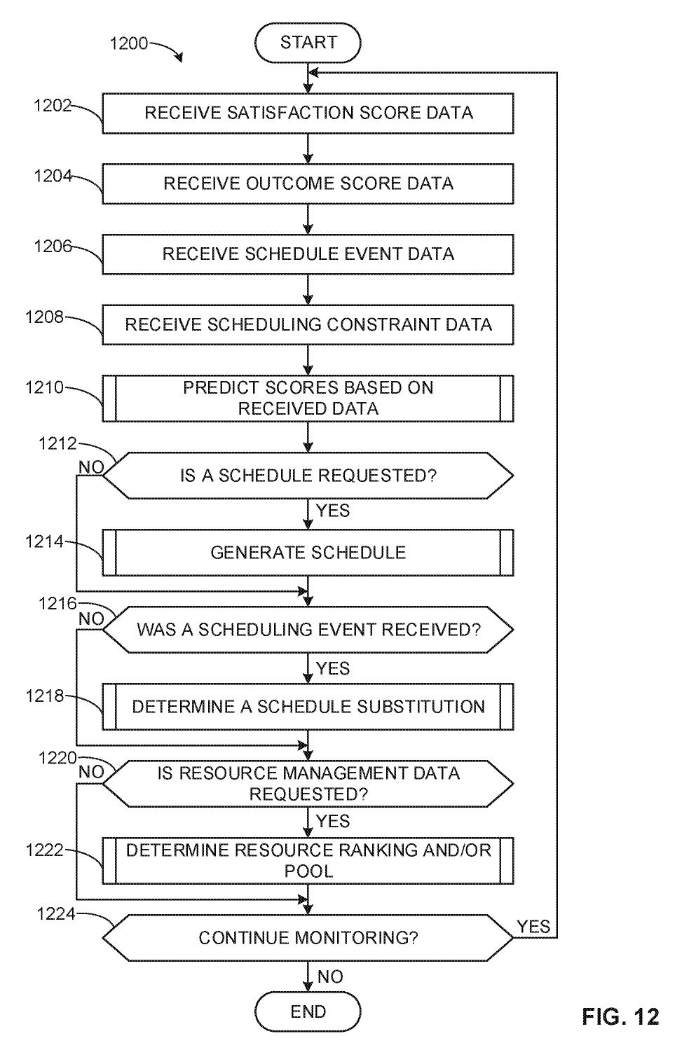

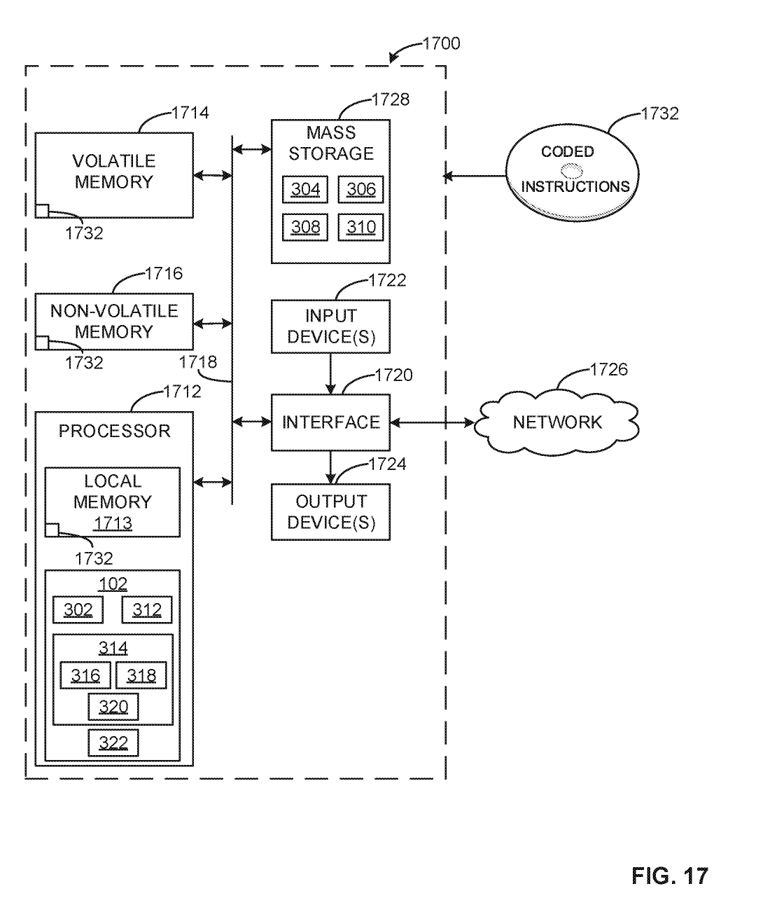

Flowcharts representative of example hardware logic or machine readable instructions for implementing the example resource director 102 of FIGS. 1A and 1B are shown in FIGS. 12-16. The machine readable instructions can be a program or portion of a program for execution by a processor such as the processor 1712 shown in the example processor platform 1700 discussed below in connection with FIG. 17. The program can be embodied in software stored on a non-transitory computer readable storage medium such as a CD-ROM, a floppy disk, a hard drive, a DVD, a Blu-ray disk, or a memory associated with the processor 1712, but the entire program and/or parts thereof can alternatively be executed by a device other than the processor 1712 and/or embodied in firmware or dedicated hardware. Further, although the example program is described with reference to the flowchart illustrated in FIGS. 12-16, many other methods of implementing the example resource director 102 can alternatively be used. For example, the order of execution of the blocks can be changed, and/or some of the blocks described can be changed, eliminated, or combined. Additionally or alternatively, any or all of the blocks can be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

As mentioned above, the example processes of FIGS. 12-16 can be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory, and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. can be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, and (6) B with C.

FIG. 12 illustrates a flow diagram of instructions for a program 1200 to cause a computer or other processor to generate a resource allocation and associated schedule. The program 1200 of FIG. 12 begins at block 1202. At blocks 1202, 1204, 1206, and 1208, the resource director 102 receives data via the network 124. More specifically, at block 1202, the resource director 102 receives patient satisfaction scores from the satisfaction survey 116 via at least one of the wearable 104, the wireless device 108, and the terminal 112 of FIG. 1A. At block 1204, the resource director 102 receives patient outcomes scores from the server 120 of FIG. 1A. In some examples, at block 1202 and block 1204, the resource director 102 is further to store patient satisfaction scores in the satisfaction score storer 308 and the patient outcome scores in the outcome score storer 306, respectively.

At block 1206, the resource director 102 receives scheduling event data from at least one of the server 120, the server 121, and the computing terminal 122 of FIG. 1A. In some examples, scheduling event data can include a scheduling request that an employee input to the computing terminal 122. Additionally or alternatively, scheduling event data can include low census and/or high census data stored in the server 120. Additionally or alternatively, scheduling event data can include employee schedule preferences stored in the server 121. In some examples, at block 1206, the resource director 102 is further to store the scheduling event in the scheduling event storer 310.

At block 1208, the resource director 102 receives scheduling constraint data from at least one of the server 121 and the computing terminal 122 of FIG. 1A. Example constraints can include, but are not limited to, hard constraints such as safe scheduling constraints including consecutive hour limits, maximum hour limits per seven-day period, and maximum number of consecutive shifts, overtime rules including hours per day and hours per week, union rules including rotation of staff, hour limits, mandatory staff rotation, etc., shift overlap conflicts, and indicated availability. Additionally, the constraints can include soft constraints such as scheduling preferences. In some examples at block 1208, the resource director 102 is further to store the scheduling constraint data in the scheduling constraint storer 304.

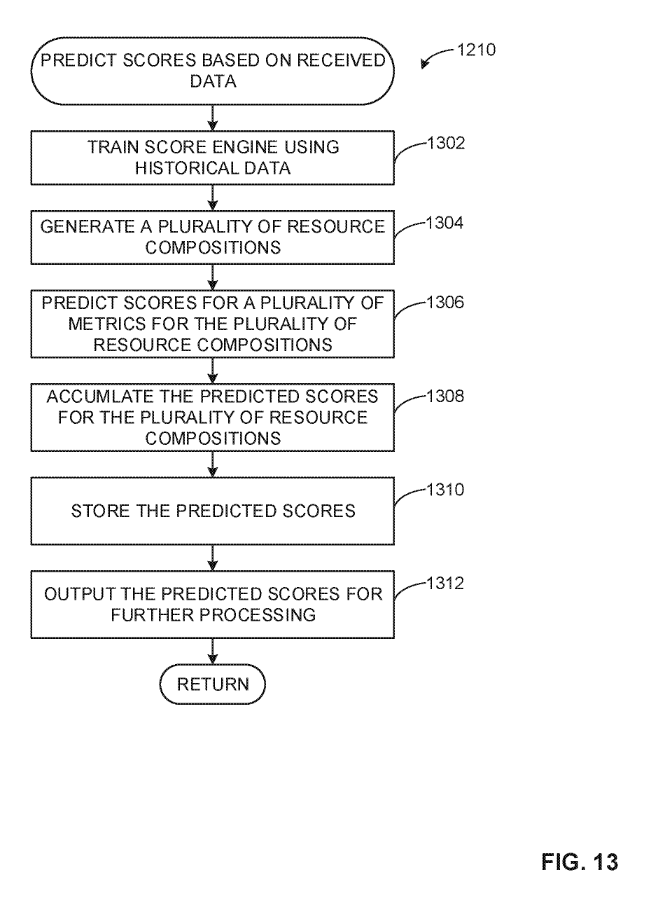

In response to receiving one or more of the datums as described in conjunction with blocks 1202-1208, processing proceeds to block 1210. At block 1210, further described in conjunction with FIG. 13, the score prediction engine 312, included in or otherwise implemented by the resource director 102, predicts scores for one or more resource compositions using machine learning techniques based on data stored in at least one of the outcome score storer 306 and the satisfaction score storer 308. Once one or more predicted resource compositions scores are generated, processing proceeds to block 1212.

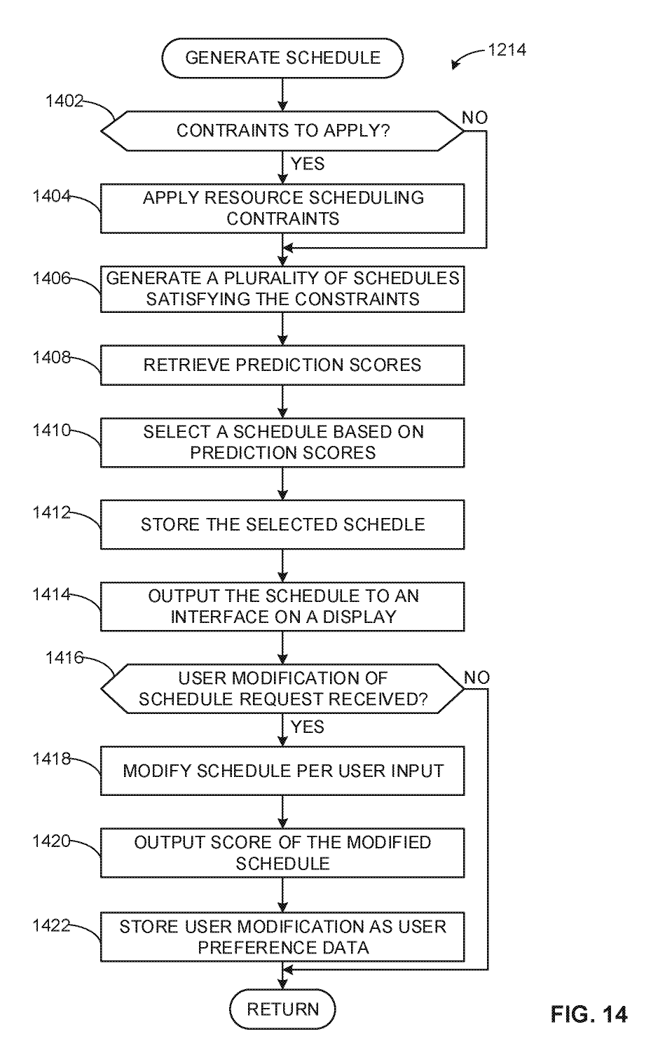

At block 1212, the resource director 102 determines whether a recommended schedule is requested. In some examples, the schedule can be automatically requested by the server 121. Additionally or alternatively, the schedule can be manually requested by a user (e.g., a manager, an administrator, a department head, etc.). In response to determining the recommended schedule is requested, processing proceeds to block 1214, further described in conjunction with FIG. 14, at which the schedule optimizer 316, included in or otherwise implemented by the resource director 102, generates a recommended schedule based on the predicted resource composition scores generated at block 1210. Conversely, in response to determining the recommended schedule is not requested, processing proceeds to block 1216.

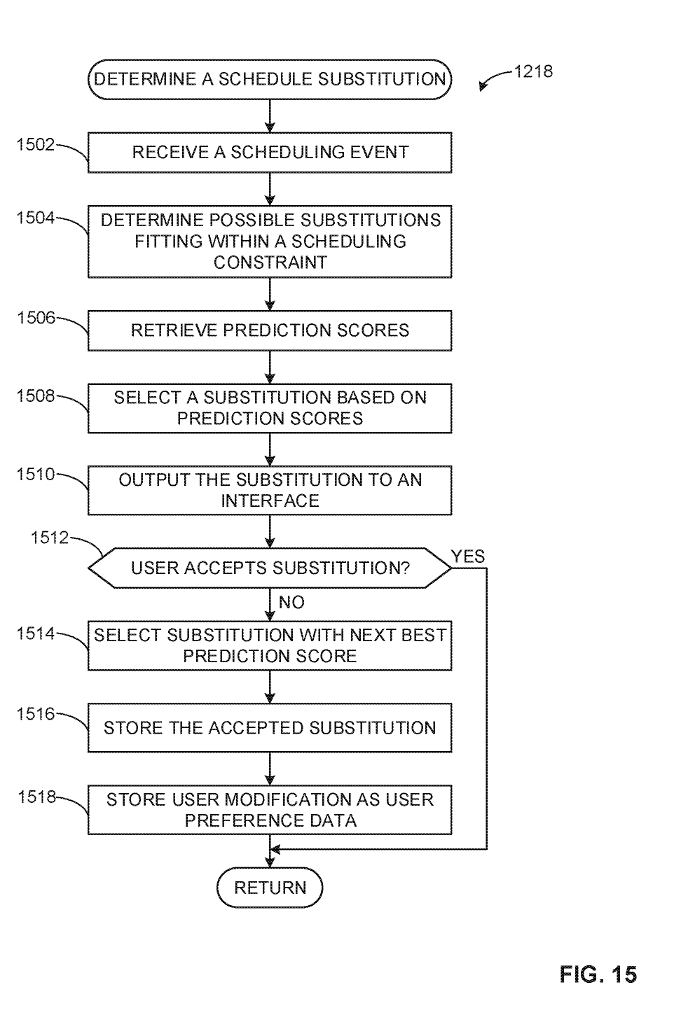

At block 1216, the resource director 102 determines whether a scheduling event was received. In some examples, the scheduling event can include a scheduling request that an employee input to the computing terminal 122. Additionally or alternatively, scheduling event data can include low census and/or high census data stored in the server 120. Additionally or alternatively, scheduling event data can include employee schedule preferences stored in the server 121. In some examples, the scheduling event may be received at the time of the scheduling event. For example, the server 120 may notify of low census/high census at the time the census event occurs. In another example, an employee may request off of work. In some examples, the scheduling event can be retrieved from the scheduling event storer 310. In response to determining the scheduling event was received, processing transfers to block 1218, further described in conjunction with FIG. 15, at which the substitution recommender 318, included in or otherwise implemented by the resource director 102, generates one or more recommended substitutions (e.g., a resource to trade based on a scheduling request, resources to send home and/or swap to a different unit based on low census, resources to call in based on high census, etc.) based on the predicted resource composition scores generated at block 1210. Conversely, in response to determining the scheduling event was not received, processing proceeds to block 1220.

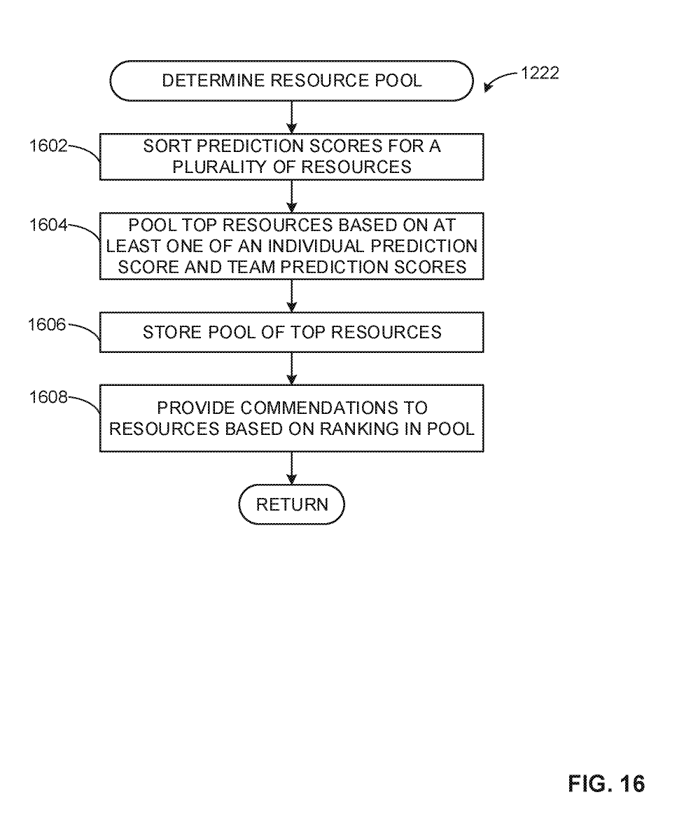

At block 1220, the resource director 102 determines whether resource management data was requested. In some examples, resource management data can be requested by at least one of the server 121 and/or the server 126 in response to an upcoming review period and/or a quantity of time elapsed since the data was previously requested. Additionally or alternatively, resource management data can be manually requested by a user. In response to determining the resource management data was requested, processing transfers to block 1222, further described in conjunction with FIG. 16, at which the resource manager 320, included in or otherwise implemented by the resource director 102, generates one or more rankings and/or pools of resources based on the predicted resource composition scores generated at block 1210. Conversely, in response to determining the resource management data was not requested, processing proceeds to block 1224.

At block 1224, the resource director 102 determines whether it is desired to continue monitoring the system 100. In response to determining the monitoring is desired, processing returns to block 1202 of the example program 1220. Conversely, in response to determining monitoring is no longer desired, the example program 1200 of FIG. 12 ends.

An example program that can be executed to predict resource composition scores based on received data (FIG. 12, block 1210) is illustrated in FIG. 13. With reference to the preceding figures and associated descriptions, the example program of FIG. 13 begins execution at block 1302, at which the neural network 402 trains the resource composition score engine 406 utilizing data stored for previous resource compositions, the data including at least one of outcome score data and satisfaction score data retrieved from the outcome score storer 306 and the satisfaction score storer 308, respectively. In some examples, training the resource composition score engine 406 with the neural network 402 can further include generating a model with the neural network 402 to be deployed with the resource composition score engine 406. In some examples, the generated model includes one or more correlation coefficients that correlate resource compositions (e.g., pairs of resources, teams of resources, nurse-doctor parings, etc.) with predicted scores. In response to deployment of the model to the resource composition score engine 406, processing proceeds to block 1304.

At block 1304, the resource composition generator 404 generates one or more resource compositions for distribution to the resource composition score engine 406, the generated resource compositions different than the resource compositions used to train the neural network 402 at block 1302. In some examples, the resource composition generator 404 adaptively generates the one or more resource compositions based on scores previously generated by the resource composition score engine 406. For example, if a certain doctor continuously appears in the highest performing resource compositions, the resource composition generator 404 can include that certain doctor in a greater portion of the generated resource compositions. In response to distribution of the one or more resource compositions to the resource composition score engine 406, processing proceeds to block 1306.

At block 1306, the resource composition score engine 406 utilizes the deployed model generated at block 1302 to predict scores for the resource compositions generated by the resource composition generator 404 at block 1304. For example, the scores can be predicted based upon a series of correlation coefficients (e.g., coefficients showing a dependence of two variables, the variables in this example being compositions of resources and outcome and/or satisfaction scores) included with the deployed model. So, for example, if the resource compositions used to train the neural network 402 include a first nurse and doctor pairing and a second nurse and doctor pairing, the resource composition generator 404 can predict a score for a pairing of the first nurse and the second doctor based upon one or more correlation coefficients. In some examples, the scores can be predicted separately as predicted outcome scores and predicted satisfaction scores.

In such examples, at block 1308, the resource composition score accumulator 408 combines the predicted outcome scores and predicted satisfaction scores into a composite predicted score. In some examples, the combination process further includes applying a weighting factor to at least one of the predicted outcome scores and the predicted satisfaction scores. In some examples, the weighting factors can be generated based on one or more user selected preferences as determined by the preference learning module 322. Additionally or alternatively, the weighting factors can be pre-determined values. In yet other examples, the weighting factors can be dynamically learned and updated by the neural network 402.

In response to completion of the combination of scores into the singular predicted scores at block 1308, the resource composition score accumulator 408 can, at block 1310, store the predicted scores in the resource composition score storer 410 and, at block 1312, output the predicted scores to the output generator 314 for use by at least one of the schedule optimizer 316, the substitution recommender 318, and the resource manager 320 for further processing. In response to completion of block 1312, the example program 1210 of FIG. 13 ends and processing returns to block 1212 of the example program 1200 of FIG. 12.

An example program that can be executed to generate a schedule (FIG. 12, block 1214) is illustrated in FIG. 14. With reference to the preceding figures and associated descriptions, the example program of FIG. 14 begins execution at block 1402 at which the constraint applicator 602 determines whether any constraints must be applied to the schedule generated by the example program 1214. Example constraints to apply can include, but are not limited to, hard constraints such as safe scheduling constraints including consecutive hour limits, maximum hour limits per 7 day period, and maximum number of consecutive shifts, overtime rules including hours per day and hours per week, union rules including rotation of staff, hour limits, mandatory staff rotation, etc., shift overlap conflicts, and indicated availability. Additionally, the constraints can include soft constraints such as scheduling preferences. In response to determining no constraints are needed and/or all constraints have been previously applied, processing proceeds to block 1406. Alternatively, in response to determining one or more constraints are unapplied, processing proceeds to block 1404 where the constraint applicator 602 applies the given constraint to a plurality of resource schedules. For example, if a candidate schedule includes an employee (e.g., a nurse, a doctor, a technician, etc.) exceeding one of the safe scheduling constraints listed above, that schedule is removed from consideration for the remainder of the example program 1214.

Once all applicable constraints are applied, the schedule generator 604 at block 1406 generates one or more resource schedules that satisfy each of the constraints applied at block 1604. Further at block 1406, the schedule generator 604 distributes the generated schedules to the schedule selector 606 and, at block 1408, the schedule selector 606 retrieves prediction scores from the score prediction engine 312.

Using the received schedules and the retrieved prediction scores associated with one or more resource compositions included in the received schedules, the schedule selector 606 is further to, at block 1410, select one of the received schedules based on which of the received schedules includes the highest prediction score for the plurality of resource compositions included in the schedule In some examples at block 1410, the schedule selector 606 can additionally or alternatively utilize one or more business objectives to determine the selected schedule. In response to selection of the schedule, the schedule selector 606 can, at block 1412, store the selected schedule in the schedule storer 608 and, at block 1414, output the selected schedule to a display. For example, the schedule selector 606 can output the selected schedule to a display associated with the computing terminal 128 of FIG. 1B for viewing by a user (e.g., a manager). In some examples, the display further includes one or more inputs, discussed further in conjunction with block 1416.

At block 1416, the schedule optimizer 316 determines whether or not the computing terminal 128 received a user input a request to modify the selected schedule. For example, the user can switch the schedules of one or more employees through the computing terminal 128, thereby modifying one or more resource compositions included in the schedule. Additionally or alternatively, the user can reject each schedule presented on the computing terminal 128 and request a plurality of different schedule options. In response to determining the user did request a modification, processing proceeds to block 1418. Alternatively, in response to determining the user did not request a modification (e.g., the user accepted the selected schedule), the example program 1214 of FIG. 14 ends and processing returns to block 1216 of the example program 1200 of FIG. 12.

At block 1418, triggered in response to a user modification, the schedule selector 606 modifies the selected schedule per the user input and determines a score for the modified schedule based on the resource composition scores previously retrieved from the score prediction engine 312 at block 1408. In some examples, the schedule selector 606 can distribute a warning to the user if the modified schedule reduces the predicted score by a threshold amount. In response to determining the score for the modified schedule, processing proceeds to block 1420 where the schedule selector 606 outputs the modified schedule and an associated score to a display such as the display associated with the computing terminal 128 of FIG. 1B. Additionally, at block 1422, the schedule selector 606 distributes the modification as a user preference (e.g., a resource removed based on the modification is non-preferred and a resource added based on the modification is preferred, etc.) for storage. In response to completion of block 1422, the example program 1214 of FIG. 14 ends and processing returns to block 1216 of the example program 1200 of FIG. 12.

An example program that can be executed to determine a scheduling substitution (FIG. 12, block 1218) is illustrated in FIG. 15. With reference to the preceding figures and associated descriptions, the example program of FIG. 15 begins execution at block 1502 at which a scheduling event is received by the event processor 702. In some examples, the scheduling event can include at least one of a scheduling request input to the computer terminal 122, a scheduling request input to the scheduling request interface 200a, a low census event retrieved from the server 120, and/or a high census event retrieved from the server 120. In some examples, at block 1502, the event processor 702 is further to determine an importance of the scheduling event (e.g., sickness and/or family leave ranked more importantly than personal time, the magnitude of the low census event and/or the high census event, etc.). In some examples, at block 1502, the event processor 702 distributes the scheduling event data to the substitution generator 704 and processing proceeds to block 1504.

At block 1504, the substitution generator 704 generates one or more schedule substitutions (e.g., a resource to shift, a resource to add, a resource to subtract, etc.) that satisfy each scheduling constraint (e.g., one or more hard constraints, one or more soft constraints, a combination of hard and soft constraints, etc.) received from the scheduling constraint storer 304. Further at block 1504, the substitution generator 704 distributes the generated schedule substitutions to the substitution selector 706 and, at block 1506, the substitution selector 706 retrieves prediction scores from the score prediction engine 312.

Using the received schedule substitutions and the retrieved prediction scores associated with one or more schedule substitutions, the substitution selector 706 is further to, at block 1508, select one of the received schedule substitutions based on which of the schedule substitutions is associated with the greatest prediction score. For example, the substitution selector 706 can add (e.g., call in) the resources with the greatest positive impact to the predicted score, subtract (e.g., send home) the resources with the greatest negative impact the predicted score, and/or trade the resource that has the greatest positive cumulative impact to each of the two or more schedules modified for the trade. In some examples, at block 1508, the substitution selector 706 can additionally or alternatively utilize one or more business objectives to determine the selected schedule substitution. In response to selection of the schedule substitution, the substitution selector 706 can, at block 1510, output the selected substitution to a display. For example, the substitution selector 706 can output the selected schedule to a display associated with the wearable 132 of FIG. 1B and/or the trade acceptance interface 200b of FIG. 2B.

At block 1512, the substitution recommender 318 determines whether or not a user input accepted or rejected the selected substitution. In some examples, the user may be a manager reviewing the score impact the substitution has. Additionally or alternatively, the user may be an employee deciding whether to accept the proposed trade through the trade acceptance interface 200b of FIG. 2B. In response to determining the user rejected the substitution, processing proceeds to block 1514. Alternatively, in response to determining the user accepted the schedule substitution, the example program 1218 of FIG. 15 ends and processing returns to block 1220 of the example program 1200 of FIG. 12.

At block 1514, triggered in response to the user rejection, the substitution selector 706 selects a second schedule substitution with a next best prediction score (e.g., the second best resource to call in, the second best resource to send home, the second best resource to trade, etc.) based on the resource composition scores previously retrieved from the score prediction engine 312 at block 1506. In response to selecting the second schedule substitution, processing proceeds to block 1516 where the substitution selector 706 stores the second schedule substitution in the substitution storer 708. Additionally, at block 1518, the substitution selector 706 distributes the modification as a user preference (e.g., a resource removed based on the modification is non-preferred and a resource added based on the modification is preferred) for storage. In response to completion of block 1518, the example program 1218 of FIG. 15 ends and processing returns to block 1220 of the example program 1200 of FIG. 12.

An example program that can be executed to determine a resource ranking and/or pool (FIG. 12, block 1222) is illustrated in FIG. 16. With reference to the preceding figures and associated descriptions, the example program of FIG. 16 begins execution at block 1602 at which the resource score sorter 802, included in or otherwise implemented by the resource manager 320, utilizes predicted resource composition scores to sort a plurality of resources by score. In some examples, the resource manager 320 can alternatively use only previous scores (e.g., non-predicted scores) to sort the plurality of resources. For example, the resources can be sorted by overall score. Additionally or alternatively, the resources can be sorted by outcome score or satisfaction score. In response to completion of the sorting of the plurality of resources, processing proceeds to block 1604.

At block 1604, a filtering technique is applied to the sorted plurality of resources determined at block 1602 by the ranked resource generator 804 to determine a pool of top resources. In some examples, the pool can be determined based on a threshold score (e.g., the pool consists of employees with a score of 90 or higher, etc.). Additionally or alternatively, the pool can be determined based on a threshold portion of a population of employees (e.g., the pool consists of the 25% of employees with the highest score, etc.). In response to completion of the pooling of resources, the ranked resource generator 804 can, at block 1606, store the pool of resources generated at block 1604 in the ranked resource storer 808. Additionally or alternatively at block 1606, the pool of resources can be stored in the server 126.

Additionally, at block 1608, the ranked resource generator 804 can provide the pooled resources to the commendation recommender 806. In some examples, resources can be commended (e.g., financial compensation, an award, etc.) as a function of respective resource scores. Additionally or alternatively, resources can be commended based on membership to one or more pools of resources. Upon completion of providing recommendations at block 1608, the example program 1222 of FIG. 16 ends and processing returns to block 1224 of the example program 1200 of FIG. 12.

FIG. 17 is a block diagram of an example processor platform 1700 structured to execute the instructions of FIGS. 12-16 to implement the resource director 102 of FIGS. 1A and 1B. The processor platform 1700 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset or other wearable device, or any other type of computing device.

The processor platform 1700 of the illustrated example includes a processor 1712. The processor 1712 of the illustrated example is hardware. For example, the processor 1712 can be implemented by one or more integrated circuits, logic circuits, microprocessors, GPUs, DSPs, or controllers from any desired family or manufacturer. The hardware processor can be a semiconductor based (e.g., silicon based) device. In this example, the processor 1712 implements the example resource director 102 which can, in some examples, include or otherwise implement the example communication manager 302, the example score prediction engine 312, the example output generator 314 which can, in some examples, include or otherwise implement the example schedule optimizer 316, the example substitution recommender 318, the example resource manager 320, and the example preference learning module 322

The processor 1712 of the illustrated example includes a local memory 1713 (e.g., a cache). The processor 1712 of the illustrated example is in communication with a main memory including a volatile memory 1714 and a non-volatile memory 1716 via a bus 1718. The volatile memory 1714 can be implemented by Synchronous Dynamic Random Access Memory (SDRAM), Dynamic Random Access Memory (DRAM), RAMBUS® Dynamic Random Access Memory (RDRAM®), and/or any other type of random access memory device. The non-volatile memory 1716 can be implemented by flash memory and/or any other desired type of memory device. Access to the main memory 1714, 1716 is controlled by a memory controller.

The processor platform 1700 of the illustrated example also includes an interface circuit 1720. The interface circuit 1720 can be implemented by any type of interface standard, such as an Ethernet interface, a universal serial bus (USB), a Bluetooth® interface, a near field communication (NFC) interface, and/or a PCI express interface.

In the illustrated example, one or more input devices 1722 are connected to the interface circuit 1720. The input device(s) 1722 permit(s) a user to enter data and/or commands into the processor 1712. The input device(s) can be implemented by, for example, an audio sensor, a microphone, a camera (still or video), a keyboard, a button, a mouse, a touchscreen, a track-pad, a trackball, an isopoint device, and/or a voice recognition system.

One or more output devices 1724 are also connected to the interface circuit 1720 of the illustrated example. The output devices 1724 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer, and/or speaker. The interface circuit 1720 of the illustrated example, thus, typically includes a graphics driver card, a graphics driver chip, and/or a graphics driver processor.

The interface circuit 1720 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 1726. The communication can be via, for example, an Ethernet connection, a digital subscriber line (DSL) connection, a telephone line connection, a coaxial cable system, a satellite system, a line-of-site wireless system, a cellular telephone system, etc.

The processor platform 1700 of the illustrated example also includes one or more mass storage devices 1728 for storing software and/or data. Examples of such mass storage devices 1728 include floppy disk drives, hard drive disks, compact disk drives, Blu-ray disk drives, redundant array of independent disks (RAID) systems, and digital versatile disk (DVD) drives.

The machine executable instructions 1732 of FIGS. 12-16 can be stored in the mass storage device 1728, in the volatile memory 1714, in the non-volatile memory 1716, and/or on a removable non-transitory computer readable storage medium such as a CD or DVD.

From the foregoing, it will be appreciated that example methods, apparatus and articles of manufacture have been disclosed that facilitate the analysis of large data flows (including data from multiple servers and/or multiple user devices) available in a hospital system to streamline the scheduling/management of resources (e.g., doctors, nurses, technicians, equipment, hospital rooms, etc.) available in a hospital. This improvement can lead to an improvement in patient and employee satisfaction as well as an improvement in patient outcomes. The examples disclosed herein can, for example, utilize the large data flows to train a neural network which generates a prediction score model which can, in some examples, include one or more correlation coefficients. The model can be used, in some examples, to predict outcome scores for resource compositions which can in turn be used in conjunction with constraints applied to the compositions to generate schedules that a) satisfy the many scheduling constraints that exist in a hospital setting and b) show the highest predicted outcome and/or satisfaction scores. In other examples, the model can be used to facilitate dynamic and real-time schedule resource substitutions (e.g., an employee trade) that have a minimal negative impact (and in some cases, a positive impact) on predicted outcomes. In yet other examples, the model can be used to predict which resources (e.g., which nurses, which doctors, etc.) have the greatest impact on the hospital system and identify said resources in a “float pool.” Thus, certain examples improve schedule processing, feedback incorporation, and processor operation to drive configuration, adjustment, and processing of hospital systems.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus comprising:
a memory to store instructions; and
a processor to be particularly programmed using the instructions to implement at least:
a score prediction engine to:
process data from the memory to determine a score for a previous resource composition; and
predict, using a trained machine learning model, a plurality of resource composition scores, the plurality of resource compositions different from the previous resource composition; and
an output generator to:
generate a plurality of results based on the plurality of resource composition scores;
select a result from the plurality of results for interactive display, the selection based upon the plurality of resource composition scores, the result including at least one of a resource schedule, a resource substitution, or a resource ranking;
output the result for interaction via an interface displayed with digital technology, the interface receiving an input to accept the result or modify the result; and
in response to receiving the input via the interface, propagate at least one of the accepted result or the modified result to a scheduling server to:
when the result includes the resource schedule, configure a first portion of the scheduling server with the result;
when the result includes the resource substitution, reconfigure the first portion of the scheduling server with the result; and
when the result includes the resource ranking, configure a second portion of the scheduling server with the result.

2. The apparatus of claim 1, wherein the score prediction engine further includes:
a neural network to train, utilizing at least one of outcome scores and satisfaction scores, a resource composition score model; and
a resource composition score engine to utilize the model developed by the neural network to predict the plurality of resource composition scores for the plurality of resource compositions.

3. The apparatus of claim 1, wherein the scheduling server is further to propagate at least one of the first portion or the second portion of the scheduling server to one or more user devices.

4. The apparatus of claim 1, wherein the output generator further includes a schedule optimizer to:
receive the plurality of resource composition scores associated with the plurality of resource compositions from the score prediction engine;
apply a set of constraints to the plurality of resource compositions to determine a plurality of resource schedules; and
select a resource schedule from the plurality of resource schedules to present based on the plurality of resource composition scores associated with the plurality of resource schedules.

5. The apparatus of claim 1, wherein the output generator further includes a substitution recommender to:
receive a scheduling event including at least one of a trade request, a low census event, or a high census event;
identify a plurality of resources capable of satisfying the scheduling event; and
select a resource from the plurality of identified resources to present based on the plurality of resource composition scores.

6. The apparatus of claim 1, wherein the output generator further includes a resource manager to:
receive a plurality of resources to be ranked by score, the score including at least one of an outcome score and a satisfaction score;
rank the plurality of resources based on respective scores, the ranking completed from a high score to a low score; and
group a portion of the plurality of resources into at least one of a high performing pool or a low performing pool, the high and low performing pools including a portion of the highest and lowest ranked resources, respectively.

7. The apparatus of claim 1, wherein the result is a first result and wherein the processor is further to implement a preference learning module to, based on the input to modify the first result, cause the output generator to apply the modification when selecting a second result to present.

8. A computer readable storage medium comprising instructions that, when executed, cause a machine to at least:
process data from a memory to determine a score for a previous resource composition;
predict, using machine learning techniques, a plurality of resource composition scores, the plurality of resource compositions different from the previous resource composition;
generate a plurality of results based on the plurality of resource composition scores
select a result from the plurality of results for interactive display, the selection based upon the plurality of resource composition scores, the result including at least one of a resource schedule, a resource substitution, or a resource ranking;
output the result for interaction via an interface displayed with digital technology, the interface receiving an input to accept the result or modify the result; and
propagate at least one of the accepted result or the modified result to a scheduling server to reconfigure one or more scheduling properties included with the server when the input is received.

9. The computer readable medium of claim 8, wherein the instructions to predict the plurality of resource composition scores further cause the machine to:
train a neural network with at least one of outcome scores and satisfaction scores to generate a resource composition score model; and
utilize the model developed by the neural network to predict the plurality of resource composition scores for the plurality of resource compositions.

10. The computer readable medium of claim 8, wherein the instruction to propagate the result further includes propagating the reconfigured scheduling properties from the scheduling server to one or more user devices.

11. The computer readable medium of claim 8, wherein the instructions to output the result, the result including the resource schedule, further cause the machine to:
receive the plurality of resource composition scores associated with the plurality of resource compositions;
apply a set of constraints to the plurality of resource compositions to determine a plurality of resource schedules; and
select the resource schedule from the plurality of resource schedules to present based on the plurality of resource composition scores associated with the plurality of resource schedules.

12. The computer readable medium of claim 8, wherein the instructions to output the result, the result including the resource substitution, further cause the machine to:
receive a scheduling event including at least one of a trade request, a low census event, or a high census event;
identify a plurality of resources capable of satisfying the scheduling event; and
select a resource as the resource substitution from the plurality of identified resources to present based on the plurality of resource composition scores.

13. The computer readable medium of claim 8, wherein the instructions to output the result, the result including the resource ranking, further cause the machine to:
receive a plurality of resources to be ranked by score, the score including at least one of an outcome score and a satisfaction score;
rank the plurality of resources based on respective scores, the ranking completed from a high score to a low score; and
group a portion of the plurality of resources into at least one of a high performing pool or a low performing pool, the high and low performing pools including a portion of the highest and lowest ranked resources, respectively.

14. The computer readable medium of claim 8, wherein the result is a first result and the instructions are further to, based on the input to modify the first result, apply the modification when selecting a second result to present.

15. A method comprising:
processing data from a memory to determine a score for a previous resource composition;
predicting, using machine learning techniques, a plurality of resource composition scores, the plurality of resource compositions different from the previous resource composition;
generating a plurality of results based on the plurality of resource composition scores;
selecting a result from the plurality of results for interactive display, the selection based upon the plurality of resource composition scores, the result including at least one of a resource schedule, a resource substitution, or a resource ranking;
outputting the result for interaction via an interface displayed with digital technology, the interface receiving an input to accept the result or modify the result; and
in response to receiving the input, propagating at least one of the accepted result or the modified result to a scheduling server to reconfigure one or more scheduling properties included with the server.

16. The method of claim 15, wherein predicting the plurality of resource composition scores further includes:
training a neural network with at least one of outcome scores and satisfaction scores to generate a resource composition score model; and
utilizing the model developed by the neural network to predict the plurality of resource composition scores for the plurality of resource compositions.

17. The method of claim 15, further including propagating the reconfigured scheduling properties from the scheduling server to one or more user devices.

18. The method of claim 15, wherein outputting the result, the result including the resource schedule, further includes:
receiving the plurality of resource composition scores associated with the plurality of resource compositions;
applying a set of constraints to the plurality of resource compositions to determine a plurality of resource schedules; and
selecting the resource schedule from the plurality of resource schedules for interactive display based on the plurality of resource composition scores associated with the plurality of resource schedules.

19. The method of claim 15, wherein outputting the result, the result including the resource substitution, further includes:
receiving a scheduling event including at least one of a trade request, a low census event, or a high census event;
identifying a plurality of resources capable of satisfying the scheduling event; and
selecting a resource as the resource substitution from the plurality of identified resources to present based on the plurality of resource composition scores.

20. The method of claim 15, wherein outputting the result, the result including the resource ranking, further includes:
receiving a plurality of resources to be ranked by score, the score including at least one of an outcome score and a satisfaction score;
ranking the plurality of resources based on respective scores, the ranking completed from a high score to a low score; and
grouping a portion of the plurality of resources into at least one of a high performing pool or a low performing pool, the high and low performing pools including a portion of the highest and lowest ranked resources, respectively.
